SEQUENCE LISTING (19 sequences)

SEQ ID NO: 1 - protein, 790 aa, unidentified
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDDTGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLDDITKTTVDFKISDKVTVKIPFSNYPSLVYTMSSKYVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 2 - RNA, 36 nt, unidentified
  source 1..36: mol_type = other RNA
gctgtgatagacctcgatttgtggggtagtaacagc

SEQ ID NO: 3 - protein, 790 aa, synthetic construct
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDDTGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLDDITKTTVDFKISDKVTVKIPFSNAPSLVYTMSSKAVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 4 - protein, 790 aa, synthetic construct
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDATGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLADITKTTVDFKISDKVTVKIPFSNAPSLVYTMSSKAVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 5 - protein, 7 aa, Betapolyomavirus macacae
PKKKRKV

SEQ ID NO: 6 - RNA, 30 nt, synthetic construct
  source 1..30: mol_type = other RNA
gtcctccttgaagtcgatgcccttcagctc

SEQ ID NO: 7 - DNA, 1041 bp, synthetic construct
  source 1..1041: mol_type = other DNA
atgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactgtcgacgccactaacttctccctgttgaaacaagcaggggatgtcgaagagaatcccgggccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 8 - RNA, 30 nt, synthetic construct
  source 1..30: mol_type = other RNA
gtagagacaggagctggagaaccatgagca

SEQ ID NO: 9 - RNA, 23 nt, synthetic construct
  source 1..23: mol_type = other RNA
gagacccagatctgccggtctct

SEQ ID NO: 10 - DNA, 1206 bp, synthetic construct
  source 1..1206: mol_type = other DNA
atgaatggcacagaaggccctaacttctacgtgcccttctccaatgcgacgggtgtggtacgcagccacttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccgtcgacgccactaacttctccctgttgaaacaagcaggggatgtcgaagagaatcccgggccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 11 - RNA, 30 nt, synthetic construct
  source 1..30: mol_type = other RNA
gaagccacctaggaccatgaagaggtcagc

SEQ ID NO: 12 - RNA, 30 nt, synthetic construct
  source 1..30: mol_type = other RNA
gcctctcttcatcctttggcccaccgtgtt

SEQ ID NO: 13 - RNA, 30 nt, synthetic construct
  source 1..30: mol_type = other RNA
gcagcttcaccttgtagatgaactcgccgt

SEQ ID NO: 14 - RNA, 30 nt, synthetic construct
  source 1..30: mol_type = other RNA
ggtcttcgatattcaagcgtcggaagacct

SEQ ID NO: 15 - RNA, 102 nt, synthetic construct
  source 1..102: mol_type = other RNA
gctgtgatagacctcgatttgtggggtagtaacagcgtcctccttgaagtcgatgcccttcagctcgctgtgatagacctcgatttgtggggtagtaacagc

SEQ ID NO: 16 - RNA, 102 nt, synthetic construct
  source 1..102: mol_type = other RNA
gctgtgatagacctcgatttgtggggtagtaacagcgtagagacaggagctggagaaccatgagcagctgtgatagacctcgatttgtggggtagtaacagc

SEQ ID NO: 17 - RNA, 102 nt, synthetic construct
  source 1..102: mol_type = other RNA
gctgtgatagacctcgatttgtggggtagtaacagcgaagccacctaggaccatgaagaggtcagcgctgtgatagacctcgatttgtggggtagtaacagc

SEQ ID NO: 18 - RNA, 102 nt, synthetic construct
  source 1..102: mol_type = other RNA
gctgtgatagacctcgatttgtggggtagtaacagcgcctctcttcatcctttggcccaccgtgttgctgtgatagacctcgatttgtggggtagtaacagc

SEQ ID NO: 19 - RNA, 102 nt, synthetic construct
  source 1..102: mol_type = other RNA
gctgtgatagacctcgatttgtggggtagtaacagcgcagcttcaccttgtagatgaactcgccgtgctgtgatagacctcgatttgtggggtagtaacagc

(19) United States
(12) Patent Application Publication
(10) Pub. No.: US 2025/0207115 A1
(43) Pub. Date: Jun. 26, 2025

(54) ENGINEERED CRISPR-CAS13F SYSTEM AND USES THEREOF

(71) Applicant: HuidaGene Therapeutics (Singapore) Pte. Ltd., Singapore (SG)

(72) Inventor: Xing Wang, Shanghai (CN)

(21) Appl. No.: 18/850,123

(22) PCT Filed: Mar. 28, 2023
(86) PCT No.: PCT/CN2023/084489

§ 371 Date: Sep. 24, 2024

(30) Foreign Application Priority Data
Mar. 28, 2022 (WO) PCT/CN2022/083461
Sep. 29, 2022 (WO) PCT/CN2022/122833

(51) Int. Cl. C12N 9/22; A61K 48/00; C12N 15/10; C12N 15/11; C12N 15/86

(52) U.S. Cl. CPC C12N 9/22; A61K 48/005; C12N 15/102; C12N 15/111; C12N 15/86; C12N 2310/20; C12N 2750/14143

(57) ABSTRACT

Provided are engineered Cas13f polypeptides, system or compositions comprising the same, and methods of using the same.

### CROSS-REFERENCE TO RELATED APPLICATIONS

This application claims the benefit of and priority to International Patent Application No. PCT/CN2022/083461, filed on Mar. 28, 2022, entitled “ENGINEERED CRISPR/CAS13 SYSTEM AND USES THEREOF”, and International Patent Application No. PCT/CN2022/122833, filed on Sep. 29, 2022, entitled “ENGINEERED CRISPR-CAS13F SYSTEM AND USES THEREOF”, which, including any sequence listing and drawings, are incorporated herein by reference in their entireties.

### REFERENCE TO AN ELECTRONIC SEQUENCE LISTING

The disclosure contains an electronic sequence listing (“HGP020PCT2. xml” created on Mar. 26, 2023, by software “WIPO Sequence” according to WIPO Standard ST. 26), which is incorporated herein by reference in its entirety. Wherever a sequence is an RNA sequence, the T in the sequence shall be deemed as U.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

Citation or identification of any document in the disclosure is not an admission that such a document is available as prior art to the disclosure.

### SUMMARY

It is against the above background that the disclosure provides certain advantages over the prior art. Although the disclosure herein is not limited to specific advantages, in an aspect, the disclosure provides an engineered Cas13f polypeptide, wherein the engineered Cas13f polypeptide:
(1) has a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.6%, 99.7%, or 99.8%) and less than 100% to the amino acid sequence of SEQ ID NO: 3;
(2) comprises a double mutation corresponding to the double mutation Y666A and Y677A of the amino acid sequence of SEQ ID NO: 3; and
(3) has an increased spacer sequence-specific cleavage activity compared to that of the amino acid sequence of SEQ ID NO: 3 and/or a decreased spacer sequence-independent collateral cleavage activity compared to that of the amino acid sequence of SEQ ID NO: 3.

In another aspect, the disclosure provides a polynucleotide encoding the engineered Cas13f polypeptide of the disclosure.

In yet another aspect, the disclosure provides a CRISPR-Cas13f system comprising:
a) the engineered Cas13f polypeptide of the disclosure or a polynucleotide (e.g., a DNA, an RNA) encoding the engineered Cas13f polypeptide; and
b) a guide nucleic acid or a polynucleotide (e.g., a DNA or an RNA) encoding the guide nucleic acid, the guide nucleic acid comprising:
i. a direct repeat (DR) sequence capable of forming a complex with the engineered Cas13f polypeptide; and,
ii. a spacer sequence capable of hybridizing to a target RNA, thereby guiding the complex to the target RNA.

In yet another aspect, the disclosure provides a vector comprising the polynucleotide of the disclosure.

In yet another aspect, the disclosure provides a delivery system comprising (1) a delivery vehicle, and (2) the engineered Cas13f polypeptide of the disclosure, the polynucleotide of the disclosure, the CRISPR-Cas13f system of the disclosure, or the vector of the disclosure.

In yet another aspect, the disclosure provides a method of modifying a target RNA, comprising contacting the target RNA with the CRISPR-Cas13f system of the disclosure, the vector of the disclosure, or the delivery system of the disclosure, thereby modifying the target RNA.

In yet another aspect, the disclosure provides a method of treating a disease in a subject in need thereof, comprising administering to the subject the CRISPR-Cas13f system of the disclosure, wherein the disease is associated with a target RNA, wherein the CRISPR-Cas13f system modifies the target RNA, and wherein the modification of the target RNA treats the disease.

With the disclosures generally described above, more detailed descriptions for the various aspects of the disclosure are provided in separate sections below. However, it should be understood that, for simplicity and to reduce redundancy, certain embodiments of the disclosure are only described under one section or only described in the claims or examples. Thus, it should also be understood that any one embodiment of the disclosure, including those described only under one aspect, section, or only in the claims or examples, can be combined with any other embodiment of the disclosure, unless specifically disclaimed or the combination is improper.

The details of one or more embodiments of the disclosure are set forth in the description below. Other features or advantages of the disclosure will be apparent from the following drawings and detailed description of several embodiments, and also from the appended claims.

### Definitions

The disclosure will be described with respect to particular embodiments, but the disclosure is not limited thereto but only by the claims. Unless defined otherwise, all technical and scientific terms used herein have the same meaning as is commonly understood by one of ordinary skill in the art to which this disclosure belongs. Terms as set forth hereinafter are generally to be understood in their common sense unless indicated otherwise.

### Overview

Class II, Type VII CRISPR-associated (Cas) protein (Cas13), as a nucleic acid programmable RNA nuclease (napRNAn), including Cas13a (C2c2), Cas13b (such as, Cas13b1, Cas13b2), Cas13c, Cas13d, Cas13e, and Cas13f polypeptides, is capable of cleaving a target RNA as guided by a guide nucleic acid (e.g., a guide RNA) comprising a guide sequence targeting the target RNA. In some embodiments, the target RNA is eukaryotic.

Without wishing to be bound by theory, in some embodiments, the guide nucleic acid comprises a scaffold sequence responsible for forming a complex with the Cas13, and a guide sequence that is intentionally designed to be responsible for hybridizing to a target sequence of the target RNA, thereby guiding the complex comprising the Cas13 and the guide nucleic acid to the target RNA.

Referring to FIG. 7, an exemplary dsRNA is depicted to comprise a 5′ to 3′ single DNA strand and a 3′ to 5′ single DNA strand. According to conventional transcription process, an exemplary RNA transcript may be transcribed using the 3′ to 5′ single DNA strand as a synthesis template, and thus the 3′ to 5′ single DNA strand is referred to as a “template strand” or a “antisense strand”. The RNA transcript so transcribed has the same primary sequence as the 5′ to 3′ single DNA strand except for the replacement of T with U, and thus the 5′ to 3′ single DNA strand is referred to as a “coding strand” or a “sense strand”.

An exemplary guide nucleic acid is depicted to comprise a guide sequence and a scaffold sequence. The guide sequence is designed to hybridize to a part of the RNA transcript (target RNA), and so the guide sequence “targets” that part. And thus, that part of the target RNA based on which the guide sequence is designed and to which the guide sequence may hybridize is referred to as a “target sequence”. In some embodiments, the guide sequence is 100% (fully) reversely complementary to the target sequence. In some other embodiments, the guide sequence is reversely complementary to the target sequence and contains a mismatch with the target sequence (as exemplified in FIG. 8).

Generally, the double-strand sequence of a dsDNA may be represented with the sequence of its 5′ to 3′ single DNA strand conventionally written in 5′ to 3′ direction/orientation. Generally, a nucleic acid sequence (e.g., a DNA sequence, an RNA sequence) is written in 5′ to 3′ direction/orientation.

For example, for a dsDNA with a 5′ to 3′ single DNA strand of 5′-ATGC-3′ and a 3′ to 5′ single DNA strand of 3′-TACG-5′, the dsDNA may be simply represented as 5′-ATGC-3′. Normally, the RNA transcript (target RNA) transcribed from the dsDNA then has a sequence of 5′-AUGC-3′.

To hybridize to the target RNA, in one embodiment, the guide sequence of a guide nucleic acid is designed to have a sequence of 5′-GCAU-3′ that is fully reversely complementary to the target RNA. According to electric sequence listing standard ST. 26 by WIPO, symbol “t” is used to denote both T in DNA and U in RNA (See “Table 1: List of nucleotides symbols”, the definition of symbol “t” is “thymine in DNA/uracil in RNA (t/u)”). Thus, in the sequence listing according to ST. 26, such a guide sequence would be set forth in GCAT but marked as an RNA sequence.

### Term

As used herein, if a DNA sequence, for example, 5′-ATGC-3′ is transcribed to an RNA sequence, with each dT (deoxythymidine, or “T” for short) in the primary sequence replaced with a U (uridine) and other dA (deoxyadenosine, or “A” for short), dG (deoxyguanosine, or “G” for short), and dC (deoxycytidine, or “C” for short) replaced with A (adenosine), G (guanosine), and C (cytidine), respectively, for example, 5′-AUGC-3′, it is said in the disclosure that the DNA sequence “encodes” the RNA sequence.

As used herein, the term “activity” refers to a biological activity. In some embodiments, the activity includes enzymatic activity, e.g., catalytic ability of an effector. For example, the activity can include nuclease activity, e.g., RNA nuclease activity, RNA endonuclease activity.

As used herein, the term “complex” refers to a grouping of two or more molecules. In some embodiments, the complex comprises a polypeptide and a nucleic acid interacting with (e.g., binding to, coming into contact with, adhering to) one another. As used herein, the term “complex” can refer to a grouping of a guide nucleic acid and a polypeptide (e.g., a napRNAn, such as, a Cas13 polypeptide). As used herein, the term “complex” can refer to a grouping of a guide nucleic acid, a polypeptide, and a target sequence. As used herein, the term “complex” can refer to a grouping of a target RNA-targeting guide nucleic acid, a napRNAn, and optionally, a target RNA.

As used herein, the term “guide nucleic acid” refers to any nucleic acid that facilitates the targeting of a napRNAn (e.g., a Cas13 polypeptide) to a target sequence (e.g., a sequence of a target RNA). A guide nucleic acid may be designed to include a sequence that is complementary to a specific nucleic acid sequence (e.g., a sequence of a target RNA). A guide nucleic acid may comprise a scaffold sequence facilitating the guiding of a napRNAn to the target RNA. In some embodiments, the guide nucleic acid is a guide RNA.

As used herein, the terms “nucleic acid”, “polynucleotide”, and “nucleotide sequence” are used interchangeably to refer to a polymeric form of nucleotides of any length, including deoxyribonucleotides, ribonucleotides, combinations thereof, and analogs or modifications thereof.

As used in the context of CRISPR-Cas techniques (e.g., CRISPR-Cas13 techniques), the term “guide RNA” is used interchangeably with the term “CRISPR RNA (crRNA)”, “single guide RNA (sgRNA)”, or “RNA guide”, the term “guide sequence” is used interchangeably with the term “spacer sequence”, and the term “scaffold sequence” is used interchangeably with the term “direct repeat sequence”.

As described herein, the guide sequence is so designed to be capable of hybridizing to a target sequence. As used herein, the term “hybridize”, “hybridizing”, or “hybridization” refers to a reaction in which one or more polynucleotide sequences react to form a complex that is stabilized via hydrogen bonding between the bases of the polynucleotide sequences. The hydrogen bonding may occur by Watson Crick base pairing, Hoogstein binding, or in any other sequence specific manner. A polynucleotide sequence capable of hybridizing to a given polynucleotide sequence is referred to as the “complement” of the given polynucleotide sequence. As used herein, the hybridization of a guide sequence and a target sequence is so stabilized to permit an effector polypeptide (e.g., a napRNAn) that is complexed with a nucleic acid comprising the guide sequence or a function domain associated (e.g., fused) with the effector polypeptide to act (e.g., cleave, deaminize) on the target sequence or its complement (e.g., a sequence of a target RNA or its complement).

For the purpose of hybridization, in some embodiments, the guide sequence is complementary or reversely complementary to a target sequence. As used herein, the term “complementary” refers to the ability of nucleobases of a first polynucleotide sequence, such as a guide sequence, to base pair with nucleobases of a second polynucleotide sequence, such as a target sequence, by traditional Watson-Crick base-pairing. Two complementary polynucleotide sequences are able to non-covalently bind under appropriate temperature and solution ionic strength conditions. In some embodiments, a first polynucleotide sequence (e.g., a guide sequence) comprises 100% (fully) complementarity to a second nucleic acid (e.g., a target sequence). In some embodiments, a first polynucleotide sequence (e.g., a guide sequence) is complementary to a second polynucleotide sequence (e.g., a target sequence) if the first polynucleotide sequence comprises at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second nucleic acid. As used herein, the term “substantially complementary” refers to a polynucleotide sequence (e.g., a guide sequence) that has a certain level of complementarity to a second polynucleotide sequence (e.g., a target sequence). In some embodiments, the level of complementarity is such that the first polynucleotide sequence (e.g., a guide sequence) can hybridize to the second polynucleotide sequence (e.g., a target sequence) with sufficient affinity to permit an effector polypeptide (e.g., a napRNAn) that is complexed with the first polynucleotide sequence or a nucleic acid comprising the first polynucleotide sequence or a function domain associated (e.g., fused) with the effector polypeptide to act (e.g., cleave, deaminize) on the target sequence or its complement (e.g., a sequence of a target RNA or its complement). In some embodiments, a guide sequence that is substantially complementary to a target sequence has less than 100% complementarity to the target sequence. In some embodiments, a guide sequence that is substantially complementary to a target sequence has at least about 80%, 81%, 82%, 83%, 84%, 85%, 86%, 87%, 88%, 89%, 90%, 91%, 92%, 93%, 94%, 95%, 96%, 97%, 98%, or 99% complementarity to the target sequence.

As used herein, the term “sequence identity” is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

Sequence homologies may be generated by any of a number of computer programs known in the art, for example BLAST or FASTA, etc. A suitable computer program for carrying out such an alignment is the GCG Wisconsin Bestfit package (University of Wisconsin, U.S.A; Devereux et al., 1984, Nucleic Acids Research 12: 387). Examples of other software than may perform sequence comparisons include, but are not limited to, the BLAST package (see Ausubel et al., 1999 ibid-Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). Percentage (%) sequence homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues. Although this is a very simple and consistent method, it fails to take into consideration that, for example, in an otherwise identical pair of sequences, one insertion or deletion may cause the following amino acid residues to be put out of alignment, thus potentially resulting in a large reduction in % homology when a global alignment is performed. Consequently, most sequence comparison methods are designed to produce optimal alignments that take into consideration possible insertions and deletions without unduly penalizing the overall homology or identity score. This is achieved by inserting “gaps” in the sequence alignment to try to maximize local homology or identity. However, these more complex methods assign “gap penalties” to each gap that occurs in the alignment so that, for the same number of identical amino acids, a sequence alignment with as few gaps as possible-reflecting higher relatedness between the two compared sequences-may achieve a higher score than one with many gaps. “Affinity gap costs” are typically used that charge a relatively high cost for the existence of a gap and a smaller penalty for each subsequent residue in the gap. This is the most commonly used gap scoring system. High gap penalties may, of course, produce optimized alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension. Calculation of maximum % homology therefore first requires the production of an optimal alignment, taking into consideration gap penalties. A new tool, called BLAST 2 Sequences is also available for comparing protein and nucleotide sequences (see FEMS Microbiol Lett. 1999 174 (2): 247-50; FEMS Microbiol Lett. 1999 177 (1): 187-8 and the website of the National Center for Biotechnology information at the website of the National Institutes for Health). Although the final % homology may be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table, if supplied (see user manual for further details). For some applications, it is preferred to use the public default values for the GCG package, or in the case of other software, the default matrix, such as BLOSUM62. Alternatively, percentage homologies may be calculated using the multiple alignment feature in DNASIS™ (Hitachi Software), based on an algorithm, analogous to CLUSTAL (Higgins D G &Sharp P M (1988), Gene 73 (1), 237-244). Once the software has produced an optimal alignment, it is possible to calculate % homology, preferably % sequence identity. The software typically does this as part of the sequence comparison and generates a numerical result. The sequences may also have deletions, insertions or substitutions of amino acid residues which produce a silent change and result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids may be grouped together based on the properties of their side chains alone. However, it is more useful to include mutation data as well. The sets of amino acids thus derived are likely to be conserved for structural reasons. These sets may be described in the form of a Venn diagram (Livingstone C. D. and Barton G. J. (1993) “Protein sequence alignments: a strategy for the hierarchical analysis of residue conservation” Comput. Appl. Biosci. 9: 745-756) (Taylor W. R. (1986) “The classification of amino acid conservation” J. Theor. Biol. 119; 205-218). Conservative substitutions may be made, for example according to the table below which describes a generally accepted Venn diagram grouping of amino acids.

As used herein, the terms “polypeptide” and “peptide” are used interchangeably herein to refer to polymers of amino acids of any length. The polymer may be linear or branched, it may comprise modified amino acids, and it may be interrupted by non-amino acids. A protein may have one or more polypeptides. The terms also encompass an amino acid polymer that has been modified; for example, by disulfide bond formation, glycosylation, lipidation, acetylation, phosphorylation, or any other manipulation, such as conjugation with a labeling component.

As used herein, a “variant” is interpreted to mean a polynucleotide or polypeptide that differs from a reference polynucleotide or polypeptide, respectively, but retains essential properties. A typical variant of a polynucleotide differs in nucleic acid sequence from another, reference polynucleotide. Changes in the nucleic acid sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

As used herein, the terms “upstream” and “downstream” refer to relative positions within a single nucleotide (e.g., DNA) sequence in a nucleic acid. Generally, a first sequence “upstream” of a second sequence means that the first sequence is 5′ to the second sequence, and a first sequence “downstream” of a second sequence means that the first sequence is 3′ to the second sequence.

As used herein, the term “wild type” has the meaning commonly understood by those skilled in the art to mean a typical form of an organism, a strain, a gene, or a feature that distinguishes it from a mutant or variant when it exists in nature. It can be isolated from sources in nature and not intentionally modified.

As used herein, the terms “non-naturally occurring” and “engineered” are used interchangeably and refer to artificial participation. When these terms are used to describe a nucleic acid or a polypeptide, it is meant that the nucleic acid or polypeptide is at least substantially freed from at least one other component of its association in nature or as found in nature.

As used herein, the “cell” is understood to refer not only to a particular individual cell, but to the progeny or potential progeny of the cell. Because certain modifications may occur in succeeding generations due to either mutation or environmental influences, such progeny may not, in fact, be identical to the parent cell, but are still included within the scope of the term.

As used herein, the term “in viva” refers to inside the body of an organism, and the terms “ex viva” or “in vitro” means outside the body of an organism.

As used herein, the term “treat”, “treatment”, or “treating” is an approach for obtaining beneficial or desired results including clinical results. For purposes of the disclosure, the beneficial or desired clinical results include, but are not limited to, one or more of the following: alleviating one or more symptoms resulting from a disease, diminishing the extent of a disease, stabilizing a disease (e.g., preventing or delaying the worsening of a disease), preventing or delaying the spread (e.g., metastasis) of a disease, preventing or delaying the recurrence of a disease, reducing recurrence rate of a disease, delay or slowing the progression of a disease, ameliorating a disease state, providing a remission (partial or total) of a disease, decreasing the dose of one or more other medications required to treat a disease, delaying the progression of a disease, increasing the quality of life, and/or prolonging survival. Also encompassed by “treatment” is a reduction of pathological consequence of a disease (such as cancer). The methods of the disclosure contemplate any one or more of these aspects of treatment.

As used herein, the term “disease” includes the terms “disorder” and “condition” and is not limited to those have been specifically medically defined.

As used herein, reference to “not” a value or parameter generally means and describes “other than” a value or parameter. For example, the method is not used to treat cancer of type X means the method may be used to treat cancer of types other than X.

As used herein, the singular forms “a”, “an”, and “the” include plural referents unless the context clearly dictates otherwise.

As used herein, the term “and/or” in a phrase such as “A and/or B” is intended to include both A and B; A or B; A (alone); and B (alone). Likewise, the term “and/or” in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, when the term “about” is ahead of a serious of numbers (for example, about 1, 2, 3), it is understood that each of the serious of numbers is modified by the term “about” (that is, about 1, about 2, about 3). The term “about X-Y” used herein has the same meaning as “about X to about Y.”

It is understood that embodiments of the disclosure described herein include “consisting” and/or “consisting essentially of” embodiments.

It is further noted that the claims may be drafted to exclude any optional element. As such, this statement is intended to serve as antecedent basis for use of such exclusive terminology as “solely”, “only”, and the like in connection with the recitation of claim elements, or use of a “negative” limitation.

### BRIEF DESCRIPTION OF THE DRAWINGS

An understanding of the features and advantages of the disclosure will be obtained by reference to the following detailed description that sets forth illustrative embodiments, in which the principles of the disclosure may be utilized, and the accompanying drawings of which:

FIG. 1 shows a view of the predicted 3D structure (by I-TASSER) of the reference Cas13f polypeptide of SEQ ID NO:1 in ribbon representation. The RXXXXH motifs of the two HEPN domains are the catalytic sites.

FIG. 2 is the schematic drawing of an exemplary one-plasmid mammalian dual-fluorescence reporter system for detecting cleavage and collateral activities of Cas13f mutants.

FIG. 3 shows 20 segments in HEPN1, HEPN2, IDL, and Hell-3 domains of reference Cas13f polypeptide of SEQ ID NO: 1 selected for mutagenesis, with each spanning 9 or 17 amino acids.

FIG. 4 is the schematic drawing of an exemplary two-plasmid mammalian dual-fluorescence reporter system for detecting cleavage and collateral activities of Cas13f mutants.

FIG. 5 shows the functional domain structure of hfCas13f. The four amino acid mutations marked in red are the mutations of hfCas13f compared with the reference Cas13f polypeptide of SEQ ID NO: 1.

FIG. 6 is the schematic drawing of an exemplary two-plasmid mammalian dual-fluorescence reporter system for detecting cleavage activities of Cas13f mutants.

FIG. 7 is a schematic showing an exemplary dsDNA, an exemplary RNA transcript transcribed from the dsDNA, an exemplary guide nucleic acid, and an exemplary Cas13, wherein the guide sequence is reversely complementary to the target sequence.

FIG. 8 is a schematic showing an exemplary dsDNA, an exemplary RNA transcript transcribed from the dsDNA, an exemplary guide nucleic acid, and an exemplary Cas13, wherein the guide sequence is reversely complementary to the target sequence and contains a mismatch with the target sequence.

The figures herein are for illustrative purposes only and are not necessarily drawn to scale.

### DETAILED DESCRIPTION

### Overview

The disclosure provides engineered Cas13f polypeptides with high cleavage activity and/or low collateral activity as desired and uses thereof.

Several subtypes of Class 2, Type VI CRISPR-associated (Cas) protein exist, including at least subtype VI-A (Cas13a/C2c2), VI-B (Cas13b1 and Cas13b2), VI-C (Cas13c), VI-D (Cas13d, CasRx), VI-E (Cas13e), and VI-F (Cas13f). The Cas13 subtypes generally share very low sequence identity/similarity, but can all be classified as Class2, Type VI Cas proteins (e.g., generally referred to herein as “Cas13”) based on the presence of two conserved HEPN-like RNase domains. Cas13 offer tremendous opportunity to knockdown target gene products (e.g., mRNA) for gene therapy, yet on the other hand, such use might be limited by its cleavage activity and/or the co-called collateral activity that poses significant risk of cytotoxicity.

For the latter, in Class 2 type VI systems, a guide sequence non-specific (independent) RNA cleavage, referred to as “collateral activity,” is conferred by the higher eukaryotes and prokaryotes nucleotide-binding (HEPN) domain in Cas13 after target RNA binding. Binding of its cognate target RNA complementary to the bound gRNA causes substantial conformational changes in Cas13, leading to the formation of a single, composite catalytic site for guide sequence non-specific “collateral” RNA cleavage, thus converting Cas13 into a guide sequence non-specific RNA nuclease. This newly formed highly accessible active site would not only degrade the target RNA in cis if the target RNA is sufficiently long to reach this new active site, but also degrade non-target RNAs in trans based on this promiscuous RNase activity. Most RNAs appear to be vulnerable to this promiscuous RNase activity of Cas13, and most (if not all) Cas13 possess this collateral activity. It has been shown recently that the collateral effect by Cas13-mediated knockdown exist in mammalian cells and animals, suggesting that clinical application of Cas13-mediated target RNA knockdown will face significant challenge in the presence of such a collateral effect.

Cas13f has been identified as a Cas13 subtype with quite a small molecular size, making it particularly suitable for delivery, e.g., by rAAV particles. In order to utilize its delivery advantage in gene therapy, it would be desirable to substantially maintain or improve its cleavage activity and/or substantially decrease or eliminate its collateral activity to prevent unwanted spontaneous cellular toxicity. Using the reporter system of the disclosure, it was found that Cas13f mutants have been developed by mutagenesis to achieve improvement in at least one aspect, or even in both aspects, of cleavage activity and collateral activity.

In some embodiments, the wild type Cas13f polypeptide of the disclosure can be: (i) SEQ ID NO: 1 (Cas13f. 1) of the disclosure, any one of SEQ ID NOs: 2-7 (Cas13f. 2, Cas13f. 3, Cas13f. 4, and Cas13f. 5, respectively) of PCT/CN2020/077211, or any one of SEQ ID NOs: 9-10 (Cas13f. 6 and Cas13f. 7, respectively) of PCT/CN2022/101884, such as SEQ ID NO: 1 of the disclosure; (ii) a naturally-occurring ortholog, paralog, or homolog of SEQ ID NO: 1 (Cas13f. 1) of the disclosure, a naturally-occurring ortholog, paralog, or homolog of any one of SEQ ID NOs: 2-7 (Cas13f. 2, Cas13f. 3, Cas13f. 4, and Cas13f. 5, respectively) of PCT/CN2020/077211, or a naturally-occurring ortholog, paralog, or homolog of any one of SEQ ID NOs: 9-10 (Cas13f. 6 and Cas13f. 7, respectively) of PCT/CN2022/101884; or (iii) a wild type Cas13 polypeptide having a sequence identity of at least about 40%, 50%, 60%, 70%, 80%, 85%, 90%, 91%, 92%, 93%, 94%, 95%, 96%, 97%, 98%, or 99% to any one of SEQ ID NO: 1 (Cas13f. 1) of the disclosure, to any one of SEQ ID NOs: 2-7 (Cas13f. 2, Cas13f. 3, Cas13f. 4, and Cas13f. 5, respectively) of PCT/CN2020/077211, to any one of SEQ ID NOs: 9-10 (Cas13f. 6 and Cas13f. 7, respectively) of PCT/CN2022/101884, or to any naturally-occurring ortholog, paralog, or homolog aforementioned.

Representative engineered Cas13f polypeptides

In an aspect, the disclosure provides an engineered Cas13f polypeptide, wherein the engineered Cas13f polypeptide:
(1) has a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.6%, 99.7%, or 99.8%) and less than 100% to the amino acid sequence of SEQ ID NO: 3;
(2) comprises a double mutation corresponding to the double mutation Y666A and Y677A of the amino acid sequence of SEQ ID NO: 3; and
(3) has an increased spacer sequence-specific cleavage activity compared to that of the amino acid sequence of SEQ ID NO: 3 and/or a decreased spacer sequence-independent collateral cleavage activity compared to that of the amino acid sequence of SEQ ID NO: 3.

In some embodiments, the engineered Cas13f polypeptide has at least about 70% (e.g., at least about 70%, 75%, 80%, 85%, 90%, 95%, 100%, 105%, 110%, 115%, 120%, 125%, 130%, 135%, 140%, 145%, or 150%) of the spacer sequence-specific cleavage activity compared to that of the amino acid sequence of SEQ ID NO: 3.

In some embodiments, the engineered Cas13f polypeptide has at most about 120% (e.g., at most about 120%, 115%, 110%, 105%, 100%, 95%, 90%, 85%, 80%, 75%, or 70%) spacer sequence-independent collateral cleavage activity compared to that of the amino acid sequence of SEQ ID NO: 3.

In some embodiments, the engineered Cas13f polypeptide has (1) at least about 75% of the spacer sequence-specific cleavage activity compared to that of the amino acid sequence of SEQ ID NO: 3, and (2) at most about 90% spacer sequence-independent collateral cleavage activity compared to that of the amino acid sequence of SEQ ID NO: 3.

In some embodiments, the engineered Cas13f polypeptide has (1) at least about 100% of the spacer sequence-specific cleavage activity compared to that of the amino acid sequence of SEQ ID NO: 3, and (2) at most about 90% spacer sequence-independent collateral cleavage activity compared to that of the amino acid sequence of SEQ ID NO: 3.

In some embodiments, the engineered Cas13f polypeptide has (1) at least about 130% of the spacer sequence-specific cleavage activity compared to that of the amino acid sequence of SEQ ID NO: 3, and (2) at most about 110% spacer sequence-independent collateral cleavage activity compared to that of the amino acid sequence of SEQ ID NO: 3.

In some embodiments, the engineered Cas13f polypeptide has (1) at least about 130% of the spacer sequence-specific cleavage activity compared to that of the amino acid sequence of SEQ ID NO: 3, and (2) at most about 100% spacer sequence-independent collateral cleavage activity compared to that of the amino acid sequence of SEQ ID NO: 3.

In some embodiments, the engineered Cas13f polypeptide has (1) at least about 130% of the spacer sequence-specific cleavage activity compared to that of the amino acid sequence of SEQ ID NO: 3, and (2) at most about 90% spacer sequence-independent collateral cleavage activity compared to that of the amino acid sequence of SEQ ID NO: 3.

In some embodiments, the engineered Cas13f polypeptide comprises an amino acid substitution at one or more positions corresponding to positions selected from the group consisting of positions 160, 161, 183, 189, 200, 202, 204, 205, 213, 214, 222, 233, 239, 240, 241, 258, 259, 276, 282, 283, 298, 299, 300, 314, 320, 329, 338, 339, 345, 353, 361, 383, 410, 433, 451, 455, 497, 508, 509, 518, 520, 526, 574, 595, 598, 599, 601, 631, 634, 638, 641, 642, 647, 667, 670, 762, 763, and combinations thereof of the amino acid sequence of SEQ ID NO: 3.

In some embodiments, the amino acid substitution is a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F), or a positively charged amino acid residue (such as, Lysine (Lys/K), Arginine (Arg/R), Histidine (His/H)).

In some embodiments, the amino acid substitution is a substitution of a non-Arginine (Arg/R) residue with an Arginine (Arg/R) residue.

In some embodiments, the amino acid substitution is a substitution of a non-Alanine (Ala/A) residue with an Alanine (Ala/A) residue.

In some embodiments, the amino acid substitution is a substitution of an Alanine (Ala/A) residue with a Valine (Val/V) residue.

In some embodiments, the engineered Cas13f polypeptide comprises an amino acid substitution at one or more positions corresponding to positions selected from the group consisting of positions 160, 161, 631, 634, 638, 641, 642, 647, 667, 670, 762, 763, and combinations thereof of the amino acid sequence of SEQ ID NO: 3.

In some embodiments, the amino acid substitution is a substitution of a non-Alanine (Ala/A) residue with an Alanine (Ala/A) residue or an Alanine (Ala/A) residue with a Valine (Val/V) residue.

In some embodiments, the engineered Cas13f polypeptide comprises an amino acid substitution with an Alanine (Ala/A) residue at one or more positions corresponding to positions selected from the group consisting of positions D 160, H638, D642, and combinations thereof of the amino acid sequence of SEQ ID NO: 3.

In some embodiments, the engineered Cas13f polypeptide comprises an amino acid substitution with an Alanine (Ala/A) residue at one or more positions corresponding to:
1) position D 160,
2) position H638,
3) position D642,
4) positions D160 and H638,
5) positions D160 &D642,
6) positions H638 &D642, or
7) positions D160 &L631,
of the amino acid sequence of SEQ ID NO: 3.

In some embodiments, the engineered Cas13f polypeptide comprises a quadruple amino acid substitution with Alanine (Ala/A) residues at positions corresponding to positions D160, D642, Y666, and Y677 of the amino acid sequence of SEQ ID NO: 1.

In some embodiments, the engineered Cas13f polypeptide comprises, consists essentially of, or consists of the amino acid sequence of SEQ ID NO: 4.

In some embodiments, the engineered Cas13f polypeptide comprises, an amino acid substitution at one or more positions corresponding to positions selected from the group consisting of positions 183, 189, 200, 202, 204, 205, 213, 214, 222, 233, 239, 240, 241, 258, 259, 276, 282, 283, 298, 299, 300, 314, 320, 329, 338, 339, 345, 353, 361, 383, 410, 433, 451, 455, 497, 508, 509, 518, 520, 526, 574, 595, 598, 599, 601, and combinations thereof of the amino acid sequence of SEQ ID NO: 3.

In some embodiments, the amino acid substitution is a substitution of a non-Arginine (Arg/R) residue with an Arginine (Arg/R) residue.

In some embodiments, the engineered Cas13f polypeptide has an increased spacer sequence-specific cleavage activity compared to that of the amino acid sequence of SEQ ID NO: 4.

In some embodiments, the engineered Cas13f polypeptide has at least about 105%, 110%, 115%, 120%, 125%, 130%, 135%, 140%, 145%, or 150% of the spacer sequence-specific cleavage activity compared to that of the amino acid sequence of SEQ ID NO: 4.

In some embodiments, the engineered Cas13f polypeptide comprises an amino acid substitution with an Arginine (Arg/R) residue at one or more positions corresponding to positions selected from the group consisting of positions G282, F314, Y338, E410, Q520, L526, F598, and combinations thereof of the amino acid sequence of SEQ ID NO: 3.

### Guide Nucleic Acid

The engineered Cas13f polypeptide of the disclosure may be used in combination with and guided by a guide nucleic acid to a target RNA to function on the target RNA.

In another aspect, the disclosure provides a guide nucleic acid comprising:
(1) a DR sequence capable of forming a complex with the engineered Cas13f polypeptide of the disclosure, and
(2) a spacer sequence capable of hybridizing to a target RNA, thereby guiding the complex to the target RNA.

In some embodiments, the spacer sequence is capable of hybridizing to a target sequence of the target RNA.

In some embodiments, the guide nucleic acid is an RNA. In some embodiments, the guide nucleic acid comprises a crRNA. In some embodiments, the guide nucleic acid does not comprise a tracrRNA.

### Structure of Guide Nucleic Acid

In some embodiments, the guide nucleic acid comprises the DR sequence 5′ or 3′ to the spacer sequence. In some embodiments, the guide nucleic acid comprises the DR sequence 3′ to the spacer sequence. In some embodiments, the DR sequence is fused to the spacer sequence without a linker.

In some embodiments, the guide nucleic acid comprises, from 5′ to 3′, one spacer sequence and one DR sequence.

In some embodiments, the guide nucleic acid comprises, from 5′ to 3′, one DR sequence, one spacer sequence, and one DR sequence, wherein the DR sequences are the same or different.

In some embodiments, the guide nucleic acid comprises, from 5′ to 3′, one DR sequence, one spacer sequence, one DR sequence, and one spacer sequence, wherein the DR sequences are the same or different, and wherein the spacer sequences are the same or different.

In some embodiments, the guide nucleic acid comprises, from 5′ to 3′, one DR sequence, one spacer sequence, one DR sequence, one spacer sequence, and one DR sequence, wherein the DR sequences are the same or different, and wherein the spacer sequences are the same or different.

In some embodiments, the guide nucleic acid comprises, from 5′ to 3′, one DR sequence, one spacer sequence, one DR sequence, one spacer sequence, one DR sequence, and one spacer sequence, wherein the DR sequences are the same or different, and wherein the spacer sequences are the same or different.

### Target RNA

The target RNA can be any RNA molecule of interest, including naturally occurring and engineered RNA molecules. The target RNA can be an mRNA, a tRNA, a ribosomal RNA (rRNA), a microRNA (miRNA), a non-coding RNA, a long non-coding (lnc) RNA, a nuclear RNA, an interfering RNA (iRNA), a small interfering RNA (siRNA), a ribozyme, a riboswitch, a satellite RNA, a microswitch, a microzyme, or a viral RNA. In some embodiments, the target RNA is a eukaryotic RNA. In some embodiments, the target RNA is encoded by a eukaryotic DNA. In some embodiments, the eukaryotic DNA is a mammal DNA, such as a non-human mammalian DNA, a non-human primate DNA, a human DNA, a plant DNA, an insect DNA, a bird DNA, a reptile DNA, a rodent (e.g., mouse, rat) DNA, a fish DNA, a nematode DNA, or a yeast DNA.

In some embodiments, the target RNA is associated with a disease (e.g., an infectious disease, a genetic disease, or a cancer). Thus, in some embodiments, the systems of the disclosure can be used to treat a disease by targeting the target RNA. For instance, the target RNA associated with a disease may be an RNA that is overexpressed in a diseased cell (e.g., a cancer or tumor cell). The target RNA may also be a toxic RNA and/or a mutated RNA (e.g., an mRNA molecule having a splicing defect or a mutation). The target RNA may also be an RNA that is specific for a particular microorganism (e.g., a pathogenic bacteria).

### Target Sequence

In some embodiments, the target sequence is at least about 14 nucleotides in length, e.g., about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides in length, or in a length of a numerical range between any of two preceding values, e.g., in a length of from about 16 to about 50 nucleotides. In some embodiments, the target sequence is about 30 nucleotides in length.

In some embodiments, the target sequence comprises, consists essentially of, or consists of at least about 14 contiguous nucleotides of a target RNA (e.g., about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of a target RNA, or in a numerical range between any of two preceding values, e.g., from about 14 to about 50 contiguous nucleotides of a target RNA). In some embodiments, the target sequence comprises, consists essentially of, or consists of about 30 contiguous nucleotides of a target RNA.

### Spacer Sequence

The spacer sequence is designed to be capable of hybridizing to the target RNA, and more specifically, to a target sequence of the target RNA. For that purpose, the primary sequence of the spacer sequence is designed to be complementary to the primary sequence of the target sequence. A 100% complementarity may not be necessary, provided that the complementarity between the spacer sequence and the target sequence is sufficient for the occurrence of the hybridization of the spacer sequence to the target sequence and the hybridization is sufficiently stable for the guiding of the engineering Cas13f polypeptide to the guide RNA by the hybridization.

In some embodiments, the spacer sequence is at least about 50%, 55%, 60%, 65%, 70%, 75%, 80%, 85%, 90%, 95%, or 100% (fully), optionally about 100% (fully), complementary to the target sequence; or wherein the spacer sequence comprises no mismatch with the target sequence in the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides at the 5′ end of the spacer sequence. It is generally believed that at least 2 mismatches between the spacer sequence and the target sequence can be tolerated for the napRNAn (e.g., Cas13) of the disclosure without significantly decreasing cleavage activity. In some embodiments, the spacer sequence is 100% (fully) complementary to the target sequence.

Typically, the spacer sequence has the same length as the target sequence. In some embodiments, the spacer sequence is at least about 14 nucleotides in length, e.g., about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides in length, or in a length of a numerical range between any of two preceding values, e.g., in a length of from about 16 to about 50 nucleotides. In some embodiments, the spacer sequence is about 30 nucleotides in length.

### DR Sequence

For the purpose of the disclosure, the DR sequence is compatible with the engineered Cas13f polypeptide of the disclosure and is capable of complexing with the engineered Cas13f polypeptide. The DR sequence may be a naturally occurring DR sequence identified along with the engineered Cas13f polypeptide, or a variant thereof maintaining the ability to complex with the engineered Cas13f polypeptide. Generally, the ability to complex with the engineered Cas13f polypeptide is maintained as long as the secondary structure of the variant is substantially identical to the secondary structure of the naturally occurring DR sequence. A nucleotide deletion, insertion, or substitution in the primary sequence of the DR sequence may not necessarily change the secondary structure of the DR sequence (e.g., the relative locations and/or sizes of the stems, bulges, and loops of the DR sequence do not significantly deviate from that of the original stems, bulges, and loops). For example, the nucleotide deletion, insertion, or substitution may be in a bulge or loop region of the DR sequence so that the overall symmetry of the bulge and hence the secondary structure remains largely the same. The nucleotide deletion, insertion, or substitution may also be in the stems of the DR sequence so that the lengths of the stems do not significantly deviate from that of the original stems (e.g., adding or deleting one base pair in each of two stems correspond to 4 total base changes).

In some embodiments, the DR sequence has substantially the same secondary structure as the secondary structure of the DR sequence of SEQ ID NO: 2.

In some embodiments, the DR sequence comprises, consists essentially of, or consists of a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 2; or a sequence having at most 1, 2, 3, 4, 5, 6, 7, 8, 9, or 10 nucleotide differences, whether consecutive or not, compared to the sequence of SEQ ID NO: 2.

In some embodiments, the DR sequence comprises the sequence of SEQ ID NO: 2.

### Modification of Guide Nucleic Acid

In some embodiments, the guide nucleic acid comprises a modification. In some embodiments, the guide nucleic acid is an unmodified RNA or modified RNA. In some embodiments, the guide nucleic acid is a modified RNA containing a modified ribonucleotide. In some embodiments, the guide nucleic acid is a modified RNA containing a deoxyribonucleotide. In some embodiments, the guide nucleic acid is a modified RNA containing a modified deoxyribonucleotide. In some embodiments, the guide nucleic acid comprises a modified or unmodified deoxyribonucleotide and a modified or unmodified ribonucleotide.

Chemical modifications can be applied to the phosphate backbone, sugar, and/or base of the guide nucleic acid. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther., 24, pp. 374-387, 2014); modifications of sugars, such as 2′-O-methyl (2′-OMe), 2′-F, and locked nucleic acid (LNA), enhance both base pairing and nuclease resistance (see, e.g., Allerson et al. “Fully 2′-modified oligonucleotide duplexes with improved in vitro potency and stability compared to unmodified small interfering RNA,” J. Med. Chem. 48.4: 901-904, 2005). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet., 2012 Aug. 20; 3: 154). Additionally, the guide nucleic acid is amenable to both 5′ and 3′ end conjugations with a variety of functional moieties including fluorescent dyes, polyethylene glycol, or proteins.

A wide variety of modifications can be applied to chemically synthesized guide nucleic acids. For example, modifying a guide nucleic acid with a 2′-OMe to improve nuclease resistance can change the binding energy of Watson-Crick base pairing. Furthermore, a 2′-OMe modification can affect how the guide nucleic acid interacts with transfection reagents, proteins or any other molecules in the cell. The effects of these modifications can be determined by empirical testing.

Examples of guide nucleic acid chemical modifications include, without limitation, incorporation of 2′-O-methyl (M), 2′-O-methyl 3′-phosphorothioate (MS), or 2′-O-methyl 3′-thioPACE (MSP) at one or more terminal nucleotides. Such chemically modified guide nucleic acids can have increased stability and/or increased activity as compared to unmodified guide nucleic acids, though on-target vs. off-target specificity is not predictable. See, Hendel, Nat Biotechnol. 33 (9): 985-9, 2015, incorporated by reference). Chemically modified guide nucleic acids may further comprise, without limitation, nucleic acids with phosphorothioate linkages and locked nucleic acid (LNA) nucleotides comprising a methylene bridge between the 2′ and 4′ carbons of the ribose ring.

In some embodiments, the guide nucleic acid comprises one or more phosphorothioate modifications. In some embodiments, the guide nucleic acid comprises one or more locked nucleic acid nucleotides for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 233: 74-83, 2016; WO 2016205764; and U.S. Pat. No. 8,795,965 B2; which is incorporated by reference in its entirety.

### Polynucleotide

In yet another aspect, the disclosure provides a polynucleotide encoding the engineered Cas13f polypeptide of the disclosure.

In some embodiments, the polynucleotide is codon optimized for expression in a eukaryote, a mammal, such as, a non-human mammal, a non-human primate, a human, a plant, an insect, a bird, a reptile, a rodent (e.g., mouse, rat), a fish, a nematode, or a yeast.

In some embodiments, the polynucleotide is a synthetic polynucleotide. In some embodiments, the polynucleotide is a DNA. In some embodiments, the polynucleotide is an RNA (e.g., an mRNA encoding the engineered Cas3f polypeptide). In some embodiments, the mRNA is capped, polyadenylated, substituted with 5-methyl cytidine, substituted with pseudouridine, or a combination thereof.

### CRISPR-Cas13f System

In yet another aspect, the disclosure provides a CRISPR-Cas13f system comprising:
a) the engineered Cas13f polypeptide of the disclosure or a polynucleotide (e.g., a DNA, an RNA) encoding the engineered Cas13f polypeptide; and
b) a guide nucleic acid or a polynucleotide (e.g., a DNA or an RNA) encoding the guide nucleic acid, the guide nucleic acid comprising:
i. a direct repeat (DR) sequence capable of forming a complex with the engineered Cas13f polypeptide; and,
ii. a spacer sequence capable of hybridizing to a target RNA, thereby guiding the complex to the target RNA.

In some embodiments, the system is a complex comprising the engineered Cas13f polypeptide complexed with the guide nucleic acid. In some embodiments, the complex further comprises the target RNA hybridized with the target sequence.

In some embodiments, the spacer sequence is capable of hybridizing to a target sequence of the target RNA.

The system of the disclosure may comprise one guide nucleic acid or more than one nucleic acid, e.g., for the purpose of improving cleavage efficiency against a target RNA.

In some embodiments, the system further comprises a second guide nucleic acid comprising:
(1) a or the DR sequence capable of forming a complex with a napRNAn, and
(2) a second spacer sequence capable of hybridizing to a second target sequence of a target RNA, thereby guiding the complex to the target RNA.

In some embodiments, the system further comprises a third guide nucleic acid comprising:
(1) a or the DR sequence capable of forming a complex with a napRNAn, and
(2) a third spacer sequence capable of hybridizing to a third target sequence of a target RNA, thereby guiding the complex to the target RNA.

In some embodiments, the system further comprises a fourth guide nucleic acid comprising:
(1) a or the DR sequence capable of forming a complex with a napRNAn, and
(2) a fourth spacer sequence capable of hybridizing to a fourth target sequence of a target RNA, thereby guiding the complex to the target RNA.

In some embodiments, the system further comprises a firth, a sixth, a seventh guide nucleic acid, and so on.

In some embodiments, the DR sequences of the more than one guide nucleic acids may be the same or slightly different (e.g., different by no more than 5, 4, 3, 2, or 1 nucleotide) to be compatible to the engineered Cas13f polypeptide.

The guide sequences of the multiple guide nucleic acids may be the same to improve cleavage activity against the same target RNA, or different to target different target RNAs in one shot.

### Regulation of Engineered Cas13f Polypeptide

In some embodiments, the polynucleotide (e.g., DNA) encoding the engineered Cas13f polypeptide of the disclosure is operably linked to a regulatory element (e.g., a promoter) in order to control the expression of the polynucleotide.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a Pglucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nP2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter, a HTT promoter, a GRK1 promoter, a CRX promoter, a NRL promoter, and a RCVRN promoter.

In some embodiments, the polynucleotide further comprises a first coding sequence for a first nuclear localization sequence (e.g., SV40 NLS, bpSV40 NLS, npNLS) or nuclear export signal (NES) 5′ to the sequence encoding the engineered Cas13 polypeptide, and/or a second coding sequence for a second NLS (e.g., SV40 NLS, bpSV40 NLS, npNLS) or NES 3′ to the sequence encoding the engineered Cas13 polypeptide.

### Regulation of Guide Nucleic Acid

In some embodiments, the polynucleotide (e.g., a DNA) encoding the guide nucleic acid is operably linked to a regulatory element (e.g., a promoter) in order to control the expression of the polynucleotide.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a Pglucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nP2 promoter, a preproenkephalin (PPE) promoter, an enkephalin (Enk) promoter, an excitatory amino acid transporter 2 (EAAT2) promoter, a glial fibrillary acidic protein (GFAP) promoter, and a myelin basic protein (MBP) promoter. In some embodiments, the promoter is a U6 promoter.

### Methods of Using

The CRISPR-Cas13f system of the disclosure comprising the engineered Cas13f polypeptide of the disclosure has a wide variety of utilities like those wild type CRISPR-Cas13 systems, including modifying (e.g., cleaving, deleting, inserting, translocating, inactivating, or activating) a target RNA in a multiplicity of cell types. The CRISPR-Cas13f systems have a broad spectrum of applications requiring high cleavage activity and low collateral activity, e.g., drug screening, disease diagnosis and prognosis, and treating various genetic disorders.

In yet another aspect, the disclosure provides a method of modifying a target RNA, comprising contacting the target RNA with the CRISPR-Cas13f system of the disclosure, the vector of the disclosure, or the delivery system of the disclosure, thereby modifying the target RNA.

In yet another aspect, the disclosure provides a method of treating a disease in a subject in need thereof, comprising administering to the subject the CRISPR-Cas13f system of the disclosure or the rAAV particle of the disclosure, wherein the disease is associated with a target RNA, wherein the CRISPR-Cas13f system modifies the target RNA, and wherein the modification of the target RNA treats the disease.

In some embodiments, the target RNA is mRNA, a tRNA, a ribosomal RNA (rRNA), a microRNA (miRNA), a non-coding RNA, a long non-coding (lnc) RNA, a nuclear RNA, an interfering RNA (iRNA), a small interfering RNA (siRNA), a ribozyme, a riboswitch, a satellite RNA, a microswitch, a microzyme, or a viral RNA.

In some embodiments, the target RNA is encoded by a eukaryotic DNA.

In some embodiments, the eukaryotic DNA is a mammal DNA, such as a non-human mammalian DNA, a non-human primate DNA, a human DNA, a plant DNA, an insect DNA, a bird DNA, a reptile DNA, a rodent (e.g., mouse, rat) DNA, a fish DNA, a nematode DNA, or a yeast DNA.

The CRISPR-Cas13f system of the disclosure can have various therapeutic applications. Such applications may be based on one or more of the abilities below, both in vitro and in vivo, attributable to the high cleavage activity of the engineered Cas13f polypeptide: cleave or degrade a target RNA, decrease or increase transcription, decrease or increase translation, inhibit or activate expression, induce or inhibit cellular senescence, induce or inhibit cell cycle arrest, induce or inhibit cell growth and/or proliferation, induce or inhibit apoptosis, induce or inhibit necrosis, etc.

In some embodiments, the CRISPR-Cas13f system can be used to treat various diseases, e.g., genetic disorders (e.g., monogenetic diseases), diseases that can be treated by RNA nuclease activity (e.g., Pcsk9 targeting, Duchenne Muscular Dystrophy (DMD) targeting, BCL1 1a targeting), and various cancers, etc.

In one aspect, the CRISPR-Cas13f system can be used for treating a disease caused by overexpression of RNAs, toxic RNAs, and/or mutated RNAs (e.g., splicing defects or truncations). For example, expression of toxic RNAs may be associated with the formation of nuclear inclusions and late-onset degenerative changes in brain, heart, or skeletal muscle. For example, in some embodiments, the disease is myotonic dystrophy. In myotonic dystrophy, the main pathogenic effect of the toxic RNAs is to sequester binding proteins and compromise the regulation of alternative splicing (see, e.g., Osbome et al., “RNA-dominant diseases,” Hum. Mol. Genet., 2009 Apr. 15; 18 (8): 1471-81). Myotonic dystrophy (dystrophia myotonica (DM)) is of particular interest to geneticists because it produces an extremely wide range of clinical features. The classical form of DM, which is now called DM type 1 (DM1), is caused by an expansion of CTG repeats in the 3′-untranslated region (UTR) of DMPK, a gene encoding a cytosolic protein kinase. The CRISPR systems as described herein can target overexpressed RNA or toxic RNA, e.g., the DMPK gene or any of the mis-regulated alternative splicing in DM1 skeletal muscle, heart, or brain.

The CRISPR-Cas13f system can also target trans-acting mutations affecting RNA-dependent functions that cause various diseases such as, e.g., Prader Willi syndrome, Spinal muscular atrophy (SMA), and Dyskeratosis congenita. A list of diseases that can be treated using the CRISPR-Cas13f system is summarized in Cooper et al., “RNA and disease,” Cell, 136.4 (2009): 777-793, and WO 2016/205764 A1, both of which are incorporated herein by reference in their entireties. Those of skill in this field will understand how to use the CRTSPR-Cas13f system to treat these diseases.

The CRISPR-Cas13f system can also be used in the treatment of various tauopathies, including, e.g., primary and secondary tauopathies, such as primary age-related tauopathy (PART)/Neurofibrillary tangle (NFT)-predominant senile dementia (with NFTs similar to those seen in Alzheimer Disease (AD), but without plaques), dementia pugilistica (chronic traumatic encephalopathy), and progressive supranuclear palsy. A useful list of tauopathies and methods of treating these diseases are described, e.g., in WO 2016205764, which is incorporated herein by reference in its entirety.

The CRISPR-Cas13f system can also be used to target mutations disrupting the cis-acting splicing codes that can cause splicing defects and diseases. These diseases include, e.g., motor neuron degenerative disease that results from deletion of the SMN1 gene (e.g., spinal muscular atrophy), Duchenne Muscular Dystrophy (DMD), frontotemporal dementia, and Parkinsonism linked to chromosome 17 (FTDP-17), and cystic fibrosis.

The CRISPR-Cas13f system can also be used for antiviral activity, in particular against RNA viruses. For example, the CRISPR-Cas13f system can be programmed with a guide nucleic acid targeting an RNA molecule associated with the RNA viruses to prevent reproduction of the RNA viruses and/or inactivate the activity of the RNA viruses.

The CRISPR-Cas13f system can also be used to treat a cancer in a subject (e.g., a human subject). For example, the CRISPR-Cas13f system can be programmed with a guide nucleic acid targeting an RNA molecule that is aberrant (e.g., comprises a point mutation or is alternatively spliced) and found in cancer cells to induce cell death of the cancer cells (e.g., via apoptosis).

The CRISPR-Cas13f system can also be used to treat an autoimmune disease or disorder in a subject (e.g., a human subject). For example, the CRISPR-Cas13f system can be programmed with a guide nucleic acid targeting an RNA molecule that is aberrant (e.g., comprises a point mutation or is alternatively spliced) and found in cells responsible for causing the autoimmune disease or disorder.

The CRISPR-Cas13f system can also be used to treat an infectious disease in a subject. For example, the CRISPR-Cas13f system can be programmed with a guide nucleic acid targeting an RNA molecule expressed by an infectious agent (e.g., a bacterium, a virus, a parasite, or a protozoan) in order to target and induce cell death in the infectious agent-containing cell. The CRISPR-Cas13f system may also be used to treat diseases where an intracellular infectious agent infects the cells of a host subject. By programming the CRISPR-Cas13f system to target a RNA molecule encoded by an infectious agent gene, cells infected with the infectious agent can be targeted and cell death induced.

A detailed description of therapeutic applications of the CRISPR-Cas13 systems described herein can be found, e.g., in U.S. Pat. No. 8,795,965, EP 3009511, WO 2016205764, and WO 2017070605; each of which is incorporated herein by reference in its entirety.

In some embodiments, the target RNA is a transcript (e.g., mRNA) of a target gene associated with an eye disease or disorder.

In some embodiments, the eye disease or disorder is amoebic keratitis, fungal keratitis, bacterial keratitis, viral keratitis, onchorcercal keratitis, keratoconjunctivitis, bacterial keratoconjunctivitis, viral keratoconjunctivitis, vernal keratoconjunctivitis, atopic keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, noninfectious uveitis, anterior uveitis, posterior uveitis (including toxoplasmosis), pan-uveitis, an inflammatory disease of the vitreous or retina, endophthalmitis prophylaxis and treatment, macular edema, macular degeneration, wet age related macular degeneration (wet AMD), dry age related macular degeneration (dry AMD), diabetic macular edema (DME), allergic conjunctivitis, proliferative and non-proliferative diabetic retinopathy, hypertensive retinopathy, an autoimmune disease of the retina, primary and metastatic intraocular melanoma, other intraocular metastatic tumors, open angle glaucoma, Stargardt's disease, Fundus Flavimaculatus, closed angle glaucoma, pigmentary glaucoma, retinitis pigmentosa (RP), Leber's congenital amaurosis (LCA), Usher's syndrome, choroideremia, a rod-cone or cone-rod dystrophy, a ciliopathy, a mitochondrial disorder, progressive retinal atrophy, a degenerative retinal disease, geographic atrophy, a familial or acquired maculopathy, a retinal photoreceptor disease, a retinal pigment epithelial-based disease, cystoid macular edema, retinal detachment, traumatic retinal injury, iatrogenic retinal injury, macular holes, macular telangiectasia, a ganglion cell disease, an optic nerve cell disease, optic neuropathy, ischemic retinal disease, retinopathy of prematurity, retinal vascular occlusion, familial macroaneurysm, a retinal vascular disease, an ocular vascular diseases, a vascular disease, an ischemic optic neuropathy disease, diabetic retinal oedema, senile macular degeneration due to sub-retinal neovascularization, myopic retinopathy, retinal ischemia, choroidal vascular insufficiency, choroidal thrombosis and neovascular retinopathies resulting from carotoid artery ischemia, corneal neovascularisation, a corneal disease or opacification with an exudative or inflammatory component, diffuse lamellar keratitis, neovascularisation due to penetration of the eye or contusive ocular injury, rubosis iritis, Fuchs' heterochromic iridocyclitis, chronic uveitis, anterior uveitis, inflammatory conditions resulting from surgeries such as LASIK, LASEK, refractive surgery, IOL implantation; irreversible corneal oedema as a complication of cataract surgery, oedema as a result of insult or trauma, inflammation, infectious and non-infectious conjunctivitis, iridocyclitis, iritis, scleritis, episcleritis, superficial punctuate keratitis, keratoconus, posterior polymorphous dystrophy, Fuch's dystrophies, aphakic and pseudophakic bullous keratopathy, corneal oedema, scleral disease, ocular cicatrcial pemphigoid, pars planitis, Posner Schlossman syndrome, Behcet's disease, Vogt-Koyanagi-Harada syndrome, hypersensitivity reactions, ocular surface disorders, conjunctival oedema, Toxoplasmosis chorioretinitis, inflammatory pseudotumor of the orbit, chemosis, conjunctival venous congestion, periorbiatal cellulits, acute dacroycystitis, non-specific vasculitis, sarcoidosis, cytomegalovirus infection, and combinations thereof.

In some embodiments, the target gene is selected from the group consisting of Vascular Endothelial Growth Factor A (VEGFA), complement factor H (CFH), age-related maculopathy susceptibility 2 (ARMS2), HtrA serine peptidase 1 (HTRA1), ATP Binding Cassette Subfamily A Member 4 (ABCA4), Peripherin-2 (PRPH2), fibulin-5 (FBLN5), ERCC Excision Repair 6 Chromatin Remodeling Factor (ERCC6), Retina And Anterior Neural Fold Homeobox 2 (RAX2), Complement C3 (C3), Toll Like Receptor 4 (TLR4), Cystatin C (CST3), CX3C Chemokine Receptor 1 (CX3CR1), complement factor I (CFI), Complement C2 (C2), Complement Factor B (CFB), Complement C9 (C9), Mitochondrially Encoded TRNA Leucine 1 (UUA/G) (MT-TL-1), Complement Factor H Related 1 (CFHR1), Complement Factor H Related 3 (CFHR3), Ciliary Neurotrophic Factor (CNTF), pigment epithelium-derived factor (PEDF), rod-derived cone viability factor (RdCVF), glial-derived neurotrophic factor (GDNF), Myosin VIIA (MYO7A); Centrosomal Protein 290 (CEP290), Cadherin Related 23 (CDH23), Eyes Shut Homolog (EYS), Usherin (USH2A), adhesion G protein-coupled receptor V1 (ADGRV1), ALMS1 Centrosome And Basal Body Associated Protein (ALMS1), Retinoid Isomerohydrolase 65 kDa (RPE65), Aryl-hydrocarbon-interacting protein-like 1 (AIPL1), Guanylate Cyclase 2D, Retinal (GUCY2D), Leber Congenital Amaurosis 5 Protein (LCA5), Cone-Rod Homeobox (CRX), Clarin (CLRN1), ATP Binding Cassette Subfamily A Member 4 (ABCA4), Retinol Dehydrogenase 12 (RDH12), Inosine Monophosphate Dehydrogenase 1 (IMPDH1), Crumbs Cell Polarity Complex Component 1 (CRB1), Lecithin retinol acyltransferase (LRAT), Nicotinamide Nucleotide Adenylyltransferase 1 (NMNAT1), TUB Like Protein 1 (TULP1), MER Proto-Oncogene, Tyrosine Kinase (MERTK), Retinitis Pigmentosa GTPase Regulato (RPGR), RP2 Activator Of ARL3 GTPase (RP2), X-linked retinitis pigmentosa GTPase regulator-interacting protein 1 (RPGRIP), Cyclic Nucleotide Gated Channel Subunit Alpha 3 (CNGA3), Cyclic Nucleotide Gated Channel Subunit Beta 3 (CNGB3), G Protein Subunit Alpha Transducin 2 (GNAT2), Fibroblast Growth Factor 2 (FGF2), Erythropoietin (EPO), BCL2 Apoptosis Regulator (BCL2), BCL2 Like 1 (BCL2L1), Nuclear Factor Kappa B (NFκB), Endostatin, Angiostatin, fms-like tyrosine kinase receptor (sFlt), Pigment-dispersing factor receptor (Pdfr), Interleukin 10 (IL10), soluble interleukin 17 (sIL17R), Interleukin-1-receptor antagonist (IL1-ra), TNF Receptor Superfamily Member 1A (TNFRSF1A), TNF Receptor Superfamily Member 1B (TNFRSF1B), and interleukin 4 (IL4).

In some embodiments, the target RNA is a transcript (e.g., mRNA) of a target gene associated with a neurodegenerative disease or disorder.

In some embodiments, the neurodegenerative disease or disorder is alcoholism, Alexander's disease, Alper's disease, Alzheimer's Disease, amyotrophic lateral sclerosis (ALS), ataxia telangiectasia, neuronal ceroid lipofuscinoses, Batten disease, bovine spongiform encephalopathy (BSE), Canavan disease, cerebral palsy, Cockayne syndrome, corticobasal degeneration, Creutzfeldt-Jakob disease, frontotemporal lobar degeneration, Huntington's disease, HIV-associated dementia, Kennedy's disease, Lewy body dementia, neuroborreliosis, primary age-related tauopathy (PART)/Neurofibrillary tangle-predominant senile dementia, Machado-Joseph disease, multiple system atrophy, multiple sclerosis, multiple sulfatase deficiency, mucolipidoses, narcolepsy, Niemann Pick disease, Parkinson's Disease, Pick's disease, Pompe disease, primary lateral sclerosis, prion diseases, neuronal loss, cognitive defect, motor neuron diseases, Duchenne Muscular Dystrophy (DMD), frontotemporal dementia, frontotemporal dementia and parkinsonism linked to chromosome 17, Lytico-Bodig disease (Parkinson-dementia complex of Guam), neuroaxonal dystrophies, Refsum's disease, Schilder's disease, subacute combined degeneration of spinal cord secondary to pernicious anaemia, Spielmeyer-Vogt-Sjogren-Batten disease, Parkinsonism linked to chromosome 17 (FTDP-17), Prader Willi syndrome, Myotonic dystrophy, chronic traumatic encephalopathy including dementia pugilistica, spinocerebellar ataxia, spinal muscular atrophy, Steele-Richardson-Olszewski disease, Tabes dorsalis, Niemann-Pick Type C (NPC1 and/or NPC2 defect), Smith-Lemli-Opitz Syndrome (SLOS), an inborn error of cholesterol synthesis, Tangier disease, Pelizaeus-Merzbacher disease, a neuronal ceroid lipofuscinosis, a primary glycosphingolipidosis, Farber disease or multiple sulphatase deficiency, Gaucher disease, Fabry disease, GM1 gangliosidosis, GM2 gangliosidosis, Krabbe disease, metachromatic leukodystrophy (MLD), NPC, GM1 gangliosidosis, Fabry disease, a neurodegenerative mucopolysaccharidosis, MPS I, MPS IH, MPS IS, MPS II, MPS III, MPS IIIA, MPS IIIB, MPS IIIC, MPS HID, MPS, IV, MPS IV A, MPS IV B, MPS VI, MPS VII, MPS IX, a disease with secondary lysosomal involvement, SLOS, Tangier disease, ganglioglioma, gangliocytoma, meningioangiomatosis, postencephalitic parkinsonism, subacute sclerosing panencephalitis, lead encephalopathy, tuberous sclerosis, Hallervorden-Spatz disease, lipofuscinosis, cerebellar ataxia, parkinsonism, Louis-Barr syndrome, multiple systems atrophy, fronto-temporal dementia or lower body Parkinson's syndrome, Niemann Pick disease, Niemann Pick type C, Niemann Pick type A, Tay-Sachs disease, multisystemic atrophy cerebellar type (MSA-C), fronto-temporal dementia with parkinsonism, progressive supranuclear palsy, cerebellar downbeat nystagmus, Sandhoff's disease or mucolipidosis type II, or combinations thereof.

In some embodiments, the target RNA is a transcript (e.g., mRNA) of a target gene associated with a cancer.

In some embodiments, the cancer is carcinomas, sarcomas, myelomas, leukemias, lymphomas and mixed type tumors. Non-limiting examples of cancers that may treated by methods and compositions described herein include, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; plasmacytoma, colorectal cancer, rectal cancer, and hairy cell leukemia.

In some embodiments, the target RNA is a transcript (e.g., mRNA) associated with a disease selected from the group consisting of: (shown in the format of “disease or disorder-causal gene or transcript”)

### Neuronal:

Rett syndrome—MECP2,
MDS-MECP2,
Angles syndrome—UBE3A-ATS,
AADC deficiency—AADC,
Canavan disease—ASPA,
Late infantile neuronal ceroid lipofuscinosis—CLN2 (also known as TPP1),
Friedreich ataxia—FRDA (also known as FXN),
Giant axonal neuropathy—GAN,
Leber's Hereditary Optic Neuropathy—ND1/ND4;

### Ocular:

Achromatopsia—CNGA3,
Leber CongenitalAmaurosis 10 Protein—CEP290,
Retinitis Pigmentosa—RHO;

### Muscular:

Dysferlinopathy—DYSF,
Danon Disease—LAMP2,
Myotonic dystrophy type 1 (DM1)—DMPK;

### Auditory:

Pendred syndrome—SLC26A4,
Wolfram syndrome—WFS1,
Stickler syndrome—COL11A2,
Nonsyndromic hearing loss—GJB2/OTOF/Myo6/STRC/KCNQ4/TECTA;

### Hepatic:

Homozygous Familial Hypercholesterolemia—LDLR/PCSK9,
Alpha—1 antitrypsin deficiency—SERPINA1;

### Others:

Phenylketonuria—phenylalanine hydroxylase (PAH),
Crigler-Najjar Syndrome—UGT1A1,
Ornithine transcarbamylase (OTC) deficiency—OTC,

### Glycogen Storage Disease Type IA-G6Pase.

In some embodiments, the disease is selected from the group consisting of glaucoma, age-related RGC loss, optic nerve injury, retinal ischemia, Leber's hereditary optic neuropathy, a neurological condition associated with degeneration of RGC neurons, a neurological condition associated with degeneration of functional neurons in the striatum of a subject in need thereof, Parkinson's disease, Alzheimer's disease, Huntington's disease, Schizophrenia, depression, drug addiction, movement disorder such as chorea, choreoathetosis, and dyskinesias, bipolar disorder, Autism spectrum disorder (ASD), dysfunction, MECP2 duplication syndrome (MDS), Angelman syndrome, age-related macular degeneration (AMD), and Amyotrophic Lateral Sclerosis (ALS).

In some embodiments, the administrating comprises local administration or systemic administration.

In some embodiments, the administrating comprises intrathecal administration, intramuscular administration, intravenous administration, transdermal administration, intranasal administration, oral administration, mucosal administration, intraperitoneal administration, intracranial administration, intracerebroventricular administration, or stereotaxic administration.

In some embodiments, the administration is conducted by injection.

In some embodiments, the subject is a human.

The dose of the rAAV particle for treatment of the disease may be either via a single dose, or multiple doses. One skilled in the art understands that the actual dose may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In some embodiments, the rAAV particle is administrated in a therapeutically effective dose. For example, the therapeutically effective dose of the rAAV particle may be about 1.0E+8, 2.0E+8, 3.0E+8, 4.0E+8, 6.0E+8, 8.0E+8, 1.0E+9, 2.0E+9, 3.0E+9, 4.0E+9, 6.0E+9, 8.0E+9, 1.0E+10, 2.0E+10, 3.0E+10, 4.0E+10, 6.0E+10, 8.0E+10, 1.0E+11, 2.0E+11, 3.0E+11, 4.0E+11, 6.0E+11, 8.0E+11, 1.0E+12, 2.0E+12, 3.0E+12, 4.0E+12, 6.0E+12, 8.0E+12, 1.0E+13, 2.0E+13, 3.0E+13, 4.0E+13, 6.0E+13, 8.0E+13, 1.0E+14, 2.0E+14, 3.0E+14, 4.0E+14, 6.0E+14, 8.0E+14, 1.0E+15, 2.0E+15, 3.0E+15, 4.0E+15, 6.0E+15, 8.0E+15, 1.0E+16, 2.0E+16, 3.0E+16, 4.0E+16, 6.0E+16, 8.0E+16, or 1.0E+17 vg, or within a range of any two of the those point values. vg stands for vector genomes of rAAV particles for administration.

In yet another aspect, the disclosure provides a pharmaceutical composition comprising the system of the disclosure or the rAAV particle of the disclosure and a pharmaceutically acceptable excipient.

In some embodiments, the pharmaceutical composition comprises the rAAV particle in a concentration selected from the group consisting of about 1×10¹⁰ vg/mL, 2×10¹⁰ vg/mL, 3×10¹⁰ vg/mL, 4×10¹⁰ vg/mL, 5×10¹⁰ vg/mL, 6×10¹⁰ vg/mL, 7×10¹⁰ vg/mL, 8×10¹⁰ vg/mL, 9×10¹⁰ vg/mL, 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹² vg/mL, 4×10¹² vg/mL, 5×10¹² vg/mL, 6×10¹² vg/mL, 7×10¹² vg/mL, 8×10¹² vg/mL, 9×10¹² vg/mL, 1×10¹³ vg/mL, or in a concentration of a numerical range between any of two preceding values, e.g., in a concentration of from about 9×10¹⁰ vg/mL to about 8×10¹¹ vg/mL.

In some embodiments, the pharmaceutical composition is an injection.

In some embodiments, the volume of the injection is selected from the group consisting of about 1 microliter, 10 microliters, 50 microliters, 100 microliters, 150 microliters, 200 microliters, 250 microliters, 300 microliters, 350 microliters, 400 microliters, 450 microliters, 500 microliters, 550 microliters, 600 microliters, 650 microliters, 700 microliters, 750 microliters, 800 microliters, 850 microliters, 900 microliters, 950 microliters, 1000 microliters, and a volume of a numerical range between any of two preceding values, e.g., in a concentration of from about 10 microliters to about 750 microliters.

### Delivery

Through this disclosure and the knowledge in the art, the CRISPR-Cas13f system of the disclosure can be delivered by various delivery systems such as vectors, e.g., plasmids, viral vectors, lipid nanoparticles (LNPs), using any suitable means in the art. Such methods include, but not limited to, electroporation, lipofection, microinjection, transfection, sonication, gene gun, etc.

One or more components of the CRISPR-Cas13f system of the disclosure, e.g., the engineered Cas13f polypeptide or a polynucleotide (e.g., a DNA, a mRNA) encoding the same, the guide nucleic acid (e.g., a gRNA) or a polynucleotide encoding the same, can be delivered using one or more suitable vectors, e.g., plasmids, viral vectors, LNPs, such as adeno-associated viruses (AAV), lentiviruses, adenoviruses, retroviral vectors, and other viral vectors, or combinations thereof. The one or more components can be packaged or encoded into one or more vectors, e.g., plasmids, viral vectors, LNPs.

The vector can be a cloning vector or an expression vector. The vectors can be plasmids, phagemids, Cosmids, etc. The vectors may include one or more regulatory elements that allow for the propagation of the vector in a cell of interest (e.g., a bacterial cell or a mammalian cell). In some embodiments, the vector comprises a polynucleotide encoding a single component of the system described herein. In some embodiments, the vector includes multiple polynucleotides, each encoding a single component of the system described herein.

In yet another aspect, the disclosure provides a vector comprising the polynucleotide of the disclosure.

In some embodiments, the polynucleotide is operably linked to a promoter. In some embodiments, the polynucleotide is operably linked to an enhancer. In some embodiments, the promoter is a constitutive promoter, an inducible promoter, a ubiquitous promoter, or a cell, tissue, or organ specific promoter, as described herein.

In some embodiments, the vector comprises a first polynucleotide encoding the engineered Cas13f polypeptide of the disclosure and a second polynucleotide encoding the guide nucleic acid of the disclosure. In some embodiments, the first and second polynucleotides are operably linked to the same promoter or separate promoters.

In some embodiments, the vector is a plasmid. In some embodiments, the delivery is via plasmids, e.g., for use in in vitro cell transfection. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding an engineered Cas13 polypeptide operably linked to (i); (iii) a selectable marker; (iv) an origin of replication; and (v) a transcription terminator downstream of and operably linked to (ii). The plasmids can further encode the guide nucleic acid of the CRISPR-Cas13f system, which may be operably linked to another promoter, to generate an all-in-one plasmid.

In some embodiments, the vector is a retroviral vector, a phage vector, an adenoviral vector, a herpes simplex viral (HSV) vector, an AAV vector, or a lentiviral vector.

In some embodiments, the AAV vector is a recombinant AAV particle comprising a capsid with a serotype of AAV1, AAV2, AAV3A, AAV3B, AAV4, AAV5, AAV6, AAV7, AAVrh74, AAV8, AAV9, AAV10, AAV11, AAV12, AAV13, AAV-DJ, AAV. PHP. eB, a member of the Clade to which any of the AAV1-AAV13 belong, or a functional variant (e.g., a functional truncation) thereof.

In some embodiments, the AAV vector is an RNA-encapsulated AAV particle.

In yet another aspect, the disclosure provides a recombinant adeno-associated virus (rAAV) vector genome comprising:
(a) a first polynucleotide sequence comprising a sequence encoding a guide nucleic acid comprising:
(1) a DR sequence capable of forming a complex with the engineered Cas13f polypeptide of the disclosure, and
(2) a spacer sequence capable of hybridizing to a target sequence of a target RNA, thereby guiding the complex to the target RNA; and
(b) a second polynucleotide sequence comprising a sequence encoding the engineered Cas13f polypeptide, wherein the rAAV vector genome is adapted to be encapsulated into a recombinant AAV particle.

Adeno-associated virus (AAV), when engineered to delivery, e.g., a protein-encoding sequence of interest, may be termed as a (r) AAV vector, a (r) AAV vector particle, or a (r) AAV particle, where “r” stands for “recombinant”. And the genome packaged in AAV vectors for delivery may be termed as a (r) AAV vector genome, vector genome, or vg for short, while viral genome may refer to the original viral genome of natural AAVs.

The serotypes of the capsids of rAAV particles can be matched to the types of target cells. For example, Table 2 of WO2018002719A1 lists exemplary cell types that can be transduced by the indicated AAV serotypes (incorporated herein by reference).

In some embodiments, the rAAV particle comprising a capsid with a serotype suitable for delivery into nerve cells (e.g., neuron). In some embodiments, the rAAV particle comprising a capsid with a serotype of AAV1, AAV2, AAV3A, AAV3B, AAV4, AAV5, AAV6, AAV7, AAVrh74, AAV8, AAV9, AAV10, AAV11, AAV12, AAV13, AAV-DJ, or AAV. PHP. eB, a member of the Clade to which any of the AAV1-AAV13 belong, or a functional variant (e.g., a functional truncation) thereof, encapsidating the rAAV vector genome. In some embodiments, the serotype of the capsid is AAV9 or AAV. PHP. eB or a mutant thereof.

General principles of rAAV particle production are known in the art. In some embodiments, rAAV particles may be produced using the triple transfection method (described in detail in U.S. Pat. No. 6,001,650).

The vector titers are usually expressed as vector genomes per ml (vg/ml). In some embodiments, the vector titer is above 1×10⁹, above 5×10¹⁰, above 1×10¹¹, above 5×10¹¹, above 1×10¹², above 5×10¹², or above 1×10¹³ vg/ml.

Instead of packaging a single strand (ss) DNA sequence as a vector genome of a rAAV particle, systems and methods of packaging an RNA sequence as a vector genome into a rAAV particle is recently developed and applicable herein. See PCT/CN2022/075366, which is incorporated herein by reference in its entirety.

When the vector genome is RNA as in, for example, PCT/CN2022/075366, for simplicity of description and claiming, sequence elements described herein for DNA vector genomes, when present in RNA vector genomes, should generally be considered to be applicable for the RNA vector genomes except that the deoxyribonucleotides in the DNA sequence are the corresponding ribonucleotides in the RNA sequence (e.g., dT is equivalent to U, and dA is equivalent to A) and/or the element in the DNA sequence is replaced with the corresponding element with a corresponding function in the RNA sequence or omitted because its function is unnecessary in the RNA sequence and/or an additional element necessary for the RNA vector genome is introduced.

As used herein, a coding sequence, e.g., as a sequence element of rAAV vector genomes herein, is construed, understood, and considered as covering and covers both a DNA coding sequence and an RNA coding sequence. When it is a DNA coding sequence, an RNA sequence can be transcribed from the DNA coding sequence, and optionally further a protein can be translated from the transcribed RNA sequence as necessary. When it is an RNA coding sequence, the RNA coding sequence per se can be a functional RNA sequence for use, or an RNA sequence can be produced from the RNA coding sequence, e.g., by RNA processing, or a protein can be translated from the RNA coding sequence.

For example, a Cas13 coding sequence encoding a Cas13 polypeptide covers either a Cas13 DNA coding sequence from which a Cas13 polypeptide is expressed (indirectly via transcription and translation) or a Cas13 RNA coding sequence from which a Cas13 polypeptide is translated (directly).

For example, a gRNA coding sequence encoding a gRNA covers either a gRNA DNA coding sequence from which a gRNA is transcribed or a gRNA RNA coding sequence (1) which per se is the functional gRNA for use, or (2) from which a gRNA is produced, e.g., by RNA processing.

In some embodiments for rAAV RNA vector genomes, 5′-ITR and/or 3′-ITR as DNA packaging signals may be unnecessary and can be omitted at least partly, while RNA packaging signals can be introduced.

In some embodiments for rAAV RNA vector genomes, a promoter to drive transcription of DNA sequences may be unnecessary and can be omitted at least partly.

In some embodiments for rAAV RNA vector genomes, a sequence encoding a polyA signal may be unnecessary and can be omitted at least partly, while a polyA tail can be introduced.

Similarly, other DNA elements of rAAV DNA vector genomes can be either omitted or replaced with corresponding RNA elements and/or additional RNA elements can be introduced, in order to adapt to the strategy of delivering an RNA vector genome by rAAV particles.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose, or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In some embodiments, the delivery is via adenoviruses, which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. The delivery methods and the doses are described, e.g., in WO 2016205764 A1 and U.S. Pat. No. 8,454,972 B2, both of which are incorporated herein by reference in the entirety.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

Various delivery methods for CRISPR-Cas13 systems are also described, e.g., in U.S. Pat. No. 8,795,965, EP 3009511, WO 2016205764, and WO 2017070605; each of which is incorporated herein by reference in its entirety.

In yet another aspect, the disclosure provides a delivery system comprising (1) a delivery vehicle, and (2) the engineered Cas13f polypeptide of the disclosure, the polynucleotide of the disclosure, the CRISPR-Cas13f system of the disclosure, or the vector of the disclosure.

In some embodiments, the delivery vehicle is a nanoparticle (e.g., LNP), a liposome, an exosome, a microvesicle, or a gene-gun.

### Cell

The methods and/or the systems of the disclosure can be used to modify of the translation and/or transcription of one or more RNA products of the cells. For example, the modification may lead to increased transcription/translation/expression of the RNA product. In other embodiments, the modification may lead to decreased transcription/translation/expression of the RNA product.

The methods of the disclosure can be used to introduce the systems described herein into a cell and cause the cell and/or its progeny to alter the production of one or more cellular produces, such as antibody, starch, ethanol, or any other desired products. Such cells and progenies thereof are within the scope of the disclosure.

In yet another aspect, the disclosure provides a cell or a progeny thereof, comprising the engineered Cas13f polypeptide of the disclosure or the system of the disclosure. In some embodiments, the cell is a eukaryote. In some embodiments, the cell is a human cell.

In yet another aspect, the disclosure provides a cell or a progeny thereof modified by the system of the disclosure or the method of the disclosure. In some embodiments, the cell is a eukaryote. In some embodiments, the cell is a human cell. In some embodiments, the cell is modified in vitro, in vivo, or ex vivo.

In some embodiments, the cell is a stem cell. In some embodiments, the cell is not a human embryonic stem cell. In some embodiments, the cell is not a human germ cell.

In some embodiments, the cell is a prokaryotic cell.

In some embodiments, the cell is a eukaryotic cell, such as a mammalian cell, including a human cell (aprimary human cell or an established human cell line). In some embodiments, the cell is a non-human mammalian cell, such as a cell from a non-human primate (e.g., monkey), a cow/bull/cattle, sheep, goat, pig, horse, dog, cat, rodent (such as rabbit, mouse, rat, hamster, etc.). In some embodiments, the cell is from fish (such as salmon), bird (such as poultry bird, including chick, duck, goose), reptile, shellfish (e.g., oyster, claim, lobster, shrimp), insect, worm, yeast, etc. In some embodiments, the cell is from a plant, such as monocot or dicot. In certain embodiment, the plant is a food crop such as barley, cassava, cotton, groundnuts or peanuts, maize, millet, oil palm fruit, potatoes, pulses, rapeseed or canola, rice, rye, sorghum, soybeans, sugar cane, sugar beets, sunflower, and wheat. In certain embodiment, the plant is a cereal (barley, maize, millet, rice, rye, sorghum, and wheat). In certain embodiment, the plant is a tuber (cassava and potatoes). In certain embodiment, the plant is a sugar crop (sugar beets and sugar cane). In certain embodiment, the plant is an oil-bearing crop (soybeans, groundnuts or peanuts, rapeseed or canola, sunflower, and oil palm fruit). In certain embodiment, the plant is a fiber crop (cotton). In certain embodiment, the plant is a tree (such as a peach or a nectarine tree, an apple or pear tree, a nut tree such as almond or walnut or pistachio tree, or a citrus tree, e.g., orange, grapefruit or lemon tree), a grass, a vegetable, a fruit, or an algae. In certain embodiment, the plant is a nightshade plant; a plant of the genus Brassica; a plant of the genus Lactuca; a plant of the genus Spinacia; a plant of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

### Kits

In yet another aspect, the disclosure provides a kit comprising the engineered Cas13f polypeptide of the disclosure, the CRISPR-Cas13f system of the disclosure, the vector of the disclosure, or the delivery system of the disclosure.

In some embodiments, the kit further comprises an instruction to use the component (s) contained therein, and/or instructions for combining with additional component (s) that may be available or necessary elsewhere.

In some embodiments, the kit further comprises one or more buffers that may be used to dissolve any of the component (s) contained therein, and/or to provide suitable reaction conditions for one or more of the component (s). Such buffers may include one or more of PBS, HEPES, Tris, MOPS, Na₂CO₃, NaHCO₃, NaB, or combinations thereof. In some embodiments, the reaction condition includes a proper pH, such as a basic pH. In some embodiments, the pH is between 7-10.

In some embodiments, any one or more of the kit components may be stored in a suitable container or at a suitable temperature, e.g., 4 Celsius degree.

Further embodiments are illustrated in the following Examples which are given for illustrative purposes only and are not intended to limit the scope of the disclosure.

The disclosure provides the following additional embodiments.

One aspect of the disclosure provides an engineered Cas13f polypeptide, wherein the engineered Cas13f polypeptide:

(1) comprises a mutation in a region spatially close to a) the N-terminal endonuclease catalytic RXXXXH motif (e.g., the N-terminal endonuclease catalytic RNFYSH motif) of a reference Cas13f polypeptide (e.g., of SEQ ID NO: 1), and/or b) the C-terminal endonuclease catalytic RXXXXH motif (e.g., the C-terminal endonuclease catalytic RNKALH motif) of the reference Cas13f polypeptide (e.g., of SEQ ID NO: 1);

(2) substantially preserves (e.g., having at least about 50%, 60%, 70%, 72.5%, 75%, 77.5%, 80%, 82.5%, 85%, 87.5%, 90%, 92.5%, 95%, 96%, 97%, 98%, 99%, or more of) the spacer sequence-specific cleavage activity of the reference Cas13f polypeptide (e.g., of SEQ ID NO: 1) towards a target RNA complementary to the spacer sequence; and

(3) substantially lacks (e.g., having no more than about 50%, 45%, 40%, 35%, 30%, 27.5%, 25%, 22.5%, 20%, 17.5%, 15%, 12.5%, 10%, 7.5%, 5%, 4.5%, 4%, 3.5%, 3%, 2.5%, 2%, 1.5%, 1% or less of) the spacer sequence-independent collateral cleavage activity of the reference Cas13f polypeptide (e.g., of SEQ ID NO: 1) towards a non-target RNA that does not bind to the spacer sequence.

In some embodiments, the region includes residues within 140, 130, 120, 110, 100, 90, 80, 70, 60, 50, 40, 30, 20, or 10 amino acids from any residues of the N-terminal endonuclease catalytic RXXXXH motif or the C-terminal endonuclease catalytic RXXXXH motif.

In some embodiments, the region includes residues more than 100, 110, 120, or 130 residues away from any residues of the N-terminal endonuclease catalytic RXXXXH motif or the C-terminal endonuclease catalytic RXXXXH motif but are spatially within about 1 to about 10 or about 5 Angstrom of any residue of the N-terminal endonuclease catalytic RXXXXH motif or the C-terminal endonuclease catalytic RXXXXH motif.

In some embodiments, the region comprises, consists essentially of, or consists of residues corresponding to the HEPN1 domain (e.g., residues 1-168), the IDL domain (e.g., residues 168-185), the Helical1 domain (e.g., Helical1-1 (Hell-1) domain (e.g., residues 185-234), Helical1-2 (Hell-2) domain (e.g., residues 281-346), Helical1-3 (Hell-3) domain (e.g., residues 477-644)), the Helical2 domain (e.g., residues 346-477), or the HEPN2 domain (e.g., residues 644-790) of the reference Cas13f polypeptide of SEQ ID NO: 1.

In some embodiments, the mutation comprises, consists essentially of, or consists of, within a stretch of about 8 to about 20 (e.g., about 9 or about 17) consecutive amino acids within the region,
(a) substitution (s) of one or more (e.g., 1, 2, 3, 4, 5, or more) non-Ala (A) residues to Ala (A) residues;
(b) substitution (s) of one or more (e.g., 1, 2, 3, 4, 5, or more) charged residues, nitrogen-containing side chain group residues, bulky (such as F or Y) residues, aliphatic residues, and/or polar residues to charge-neutral short chain aliphatic residues (such as A, V, or I);
(c) substitution (s) of one or more (e.g., 1, 2, 3, 4, 5, or more) Ile (I) and/or Leu (L) residues to Ala (A) residues; and/or
(d) substitution (s) of one or more (e.g., 1, 2, 3, 4, 5, or more) Ala (A) residues to Val (V) residues.

In some embodiments, the one or more non-Ala residues and/or the one or more charged or polar residues comprise N, Q, R, K, H, D, E, Y, S, T, L residues or a combination thereof.

In some embodiments, the one or more non-Ala residues and/or the one or more charged or polar residues comprise N, Q, R, K, H, D, Y, L residues or a combination thereof.

In some embodiments, one or more Y residue (s) within the stretch is substituted.

In some embodiments, the one or more Y residues (s) correspond to Y666 and/or Y677 of the reference Cas13f polypeptide of SEQ ID NO: 1.

In some embodiments, one or more D residue (s) within the stretch is substituted.

In some embodiments, the one or more D residues (s) correspond to D160 and/or D642 of the reference Cas13f polypeptide of SEQ ID NO: 1.

In some embodiments, the charge-neutral short chain aliphatic residue is Ala (A).

In some embodiments, the mutation comprises, consists essentially of, or consists of:
(a) substitutions within 1, 2, 3, 4, or 5 of the stretches of about 8 to about 20 (e.g., about 9 or about 17) consecutive amino acids within the region;
(b) a mutation corresponding to a mutation (e.g., any one in Tables 1-4) that results in an engineered Cas13f polypeptide having at least about 75% of a spacer sequence-specific cleavage activity and no more than about 25% of a spacer sequence-independent collateral cleavage activity, or a combination thereof; and/or
(c) a mutation corresponding to the F7V2, F10V1, F10V4, F40V4, F40S22, F40S26, F40S36, F10S21, F10S24, F10S26, F10S27, F10S33, F10S34, F10S35, F10S36, F10S45, F10S46, F10S48, F10S49, F40S23, or F40S27 mutation in Table 1-4, or a combination thereof.

In some embodiments, the engineered Cas13f polypeptide retains at least about 50%, 60%, 70%, 72.5%, 75%, 77.5%, 80%, 82.5%, 85%, 87.5%, 90%, 92.5%, 95%, 96%, 97%, 98%, 99%, or more of the spacer sequence-specific cleavage activity of the reference Cas13f polypeptide of SEQ ID NO: 1 towards the target RNA.

In some embodiments, the engineered Cas13f polypeptide has no more than 50%, 45%, 40%, 35%, 30%, 27.5%, 25%, 22.5%, 20%, 17.5%, 15%, 12.5%, 10%, 7.5%, 5%, 4.5%, 4%, 3.5%, 3%, 2.5%, 2%, 1.5%, 1%, or less of the spacer sequence-independent collateral cleavage activity of the reference Cas13f polypeptide of SEQ ID NO: 1 towards the non-target RNA.

In some embodiments, the engineered Cas13f polypeptide has at least about 80% of the spacer sequence-specific cleavage activity of the reference Cas13f polypeptide of SEQ ID NO: 1 towards the target RNA and no more than about 40% of the spacer sequence-independent collateral cleavage activity of the reference Cas13f polypeptide of SEQ ID NO: 1 towards the non-target RNA.

In some embodiments, the mutation is F40S23 (i.e., Y666A/Y677A double mutation).

In some embodiments, the engineered Cas13f polypeptide comprises, consists essentially of, or consists of the amino acid sequence of SEQ ID NO: 3.

In some embodiments, the engineered Cas13f polypeptide further comprises a mutation corresponding to a combination of any one, two, or more (e.g., 3, 4, or 5 more) mutations in Table 5 (such as, D160A, D642A, and/or L641A).

In some embodiments, the mutation is a combination of any one, two, or more (e.g., 3, 4, or 5 more) single mutations in Table 5 (such as, D 160A, D642A, and/or L64 1A) with F40S23 (i.e., Y666A/Y677A double mutation).

In some embodiments, the mutation is a Y666A/Y677A double mutation in combination with 1, 2, or 3 mutations selected from D160A, L641A, and D642A.

In some embodiments, the mutation is any combination mutations in Tables 6-11.

In some embodiments, the mutation is a D 160A/D642A/Y666A/Y677A quadruple mutation.

In some embodiments, the engineered Cas13f polypeptide has increased spacer sequence-specific cleavage activity than that of the engineered Cas13f polypeptide of SEQ ID NO: 3.

In some embodiments, the mutation is a mutation corresponding to a combination of a mutation in Tables 12-15 with D 160A/D642A/Y666A/Y677A mutation.

In some embodiments, the engineered Cas13f polypeptide comprises, consists essentially of, or consists of the amino acid sequence of SEQ ID NO: 4.

In some embodiments, the engineered Cas13f polypeptide further comprises an amino acid substitution of a non-basic amino acid residue to Arg (R) residue.

In some embodiments, the engineered Cas13f polypeptide further comprises a mutation corresponding to a combination of any one, two, or more (e.g., 3, 4, or 5 more) single mutations in Tables 12-15.

In some embodiments, the engineered Cas13f polypeptide has increased spacer sequence-specific cleavage activity than that of the engineered Cas13f polypeptide of SEQ ID NO: 4.

In some embodiments, the engineered Cas13f polypeptide has a sequence identity of at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% and less than 100% to the reference Cas13f polypeptide of SEQ ID NO: 1.

In some embodiments, the engineered Cas13f polypeptide further comprises a nuclear localization signal (NLS) sequence or a nuclear export signal (NES).

In some embodiments, the engineered Cas13f polypeptide further comprises an N- and/or a C-terminal NLS.

Another aspect of the disclosure provides a polynucleotide encoding the engineered Cas13f polypeptide of the disclosure.

In some embodiments, the polynucleotide is codon-optimized for expression in a eukaryote, a mammal, such as a human or a non-human mammal, a plant, an insect, a bird, a reptile, a rodent (e.g., mouse, rat), a fish, a worm/nematode, or a yeast.

Another aspect of the disclosure provides a CRISPR-Cas13f system comprising:
a) the engineered Cas13f polypeptide of the disclosure or a polynucleotide coding sequence (e.g., a DNA coding sequence or an RNA coding sequence) thereof; and
b) a guide RNA (gRNA) or a polynucleotide coding sequence (e.g., a DNA coding sequence or an RNA coding sequence) thereof, the gRNA comprising:
i. a direct repeat (DR) sequence capable of forming a complex with the engineered Cas13f polypeptide; and,
ii. a spacer sequence capable of hybridizing to a target RNA, and guiding or recruiting the complex to the target RNA.

In some embodiments, the DR sequence has substantially the same secondary structure of that of SEQ ID NO: 2.

In some embodiments, the spacer sequence is in a length of at least 15 nucleotides. In some embodiments, the spacer sequence is in a length of 30 nucleotides.

Another aspect of the disclosure provides a vector comprising the polynucleotide of the disclosure.

In some embodiments, the polynucleotide is operably linked to a promoter. In some embodiments, the polynucleotide is operably linked to an enhancer.

In some embodiments, the promoter is a constitutive promoter, an inducible promoter, a ubiquitous promoter, or a cell, tissue, or organ specific promoter.

In some embodiments, the vector is a plasmid.

In some embodiments, the vector is a retroviral vector, a phage vector, an adenoviral vector, a herpes simplex viral (HSV) vector, an AAV vector, or a lentiviral vector.

In some embodiments, the AAV vector is a recombinant AAV vector of the serotype AAV1, AAV2, AAV4, AAV5, AAV6, AAV7, AAVrh74, AAV8, AAV9, AAV10, AAV 11, AAV 12, AAV 13, AAV. PHP. eB, or AAV-DJ.

In some embodiments, the AAV vector is an RNA-encapsulated AAV vector.

Another aspect of the disclosure provides a delivery system comprising (1) a delivery vehicle, and (2) the engineered Cas13f polypeptide of the disclosure, the polynucleotide of the disclosure, CRISPR-Cas13f system of the disclosure, or the vector of the disclosure.

In some embodiments, the delivery vehicle is a nanoparticle (e.g., LNP), a liposome, an exosome, a microvesicle, or a gene-gun.

Another aspect of the disclosure provides a cell or a progeny thereof, comprising the engineered Cas13f polypeptide of the disclosure, the polynucleotide of the disclosure, CRISPR-Cas13f system of the disclosure, the vector of the disclosure, or the delivery system of the disclosure.

In some embodiments, the cell is a eukaryotic cell (e.g., a non-human mammalian cell, a human cell, or a plant cell) or a prokaryotic cell (e.g., a bacteria cell).

Another aspect of the disclosure provides a non-human multicellular eukaryote comprising the cell or progeny of the disclosure.

In some embodiments, the non-human multicellular eukaryote is an animal (e.g., rodent or primate) model for a human genetic disorder.

Another aspect of the disclosure provides a method of modifying a target RNA, the method comprising contacting the target RNA with the CRISPR-Cas13f system of the disclosure, the vector of the disclosure, the delivery system of the disclosure, or the cell or progeny of the disclosure.

In some embodiments, the target RNA is modified by cleavage by the engineered Cas13f polypeptide.

In some embodiments, the target RNA is an mRNA, a tRNA, an rRNA, a non-coding RNA, a lncRNA, or a nuclear RNA.

In some embodiments, upon binding of the complex of the engineered Cas13f polypeptide and the guide RNA to the target RNA, the engineered Cas13f polypeptide does not exhibit substantial (or detectable) spacer sequence-independent collateral cleavage activity.

In some embodiments, the target RNA is within a cell.

In some embodiments, the cell is a cancer cell.

In some embodiments, the cell is infected with an infectious agent.

In some embodiments, the infectious agent is a virus, a prion, a protozoan, a fungus, or a parasite.

In some embodiments, the cell is a neuronal cell (e.g., astrocyte, glial cell (e.g., Muller glia cell, oligodendrocyte, ependymal cell, Schwan cell, NG2 cell, or satellite cell)).

In some embodiments, the CRISPR-Cas13f system is encoded by a first polynucleotide encoding the engineered Cas13f polypeptide, and a second polynucleotide comprising or encoding the guide RNA, wherein the first and the second polynucleotides are introduced into the cell.

In some embodiments, the first and the second polynucleotides are introduced into the cell by the same vector.

In some embodiments, the contacting causes one or more of: (i) in vitro or in vivo induction of cellular senescence; (ii) in vitro or in vivo cell cycle arrest; (iii) in vitro or in vivo cell growth inhibition; (iv) in vitro or in vivo induction of anergy; (v) in vitro or in vivo induction of apoptosis; and (vi) in vitro or in vivo induction of necrosis.

Another aspect of the disclosure provides a method of treating a disease in a subject in need thereof, the method comprising administering to the subject a composition comprising the CRISPR-Cas13f system of the disclosure, the vector of the disclosure, the delivery system of the disclosure, or the cell or progeny of the disclosure; wherein upon administrating, the engineered Cas13f polypeptide cleaves the target RNA, thereby treating the disease in the subject.

In some embodiments, the disease is a neurological condition, a cancer, an infectious disease, or a genetic disorder.

In some embodiments, the cancer is Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

In some embodiments, the neurological condition is glaucoma, age-related RGC loss, optic nerve injury, retinal ischemia, Leber's hereditary optic neuropathy, a neurological condition associated with degeneration of RGC neurons, a neurological condition associated with degeneration of functional neurons in the striatum of a subject in need thereof, Parkinson's disease, Alzheimer's disease, Huntington's disease, Schizophrenia, depression, drug addiction, movement disorder such as chorea, choreoathetosis, and dyskinesias, bipolar disorder, Autism spectrum disorder (ASD), or dysfunction.

In some embodiments, the method is an in vitro method, an in vivo method, or an ex vivo method.

Another aspect of the disclosure provides a CRISPR-Cas13f complex comprising the engineered Cas13f polypeptide of the disclosure, and a guide RNA comprising a DR sequence that binds the engineered Cas13f polypeptide and a spacer sequence capable of hybridizing to a target RNA, and guiding or recruiting the complex to the target RNA.

In some embodiments, the target RNA is encoded by a eukaryotic DNA.

In some embodiments, the eukaryotic DNA is a non-human mammalian DNA, a non-human primate DNA, a human DNA, a plant DNA, an insect DNA, a bird DNA, a reptile DNA, a rodent DNA, a fish DNA, a worm/nematode DNA, or a yeast DNA.

In some embodiments, the target RNA is an mRNA.

In some embodiments, the CRISPR-Cas13f complex further comprises a target RNA comprising a sequence capable of hybridizing to the spacer sequence.

### EXAMPLES

The following examples are provided to further illustrate some embodiments of the disclosure but are not intended to limit the scope of the invention; it will be understood by their exemplary nature that other procedures, methodologies, or techniques known to those skilled in the art may alternatively be used.

### Example 1 Engineering of Reference Cas13f Polypeptide for Collateral Activity

This Example demonstrates that by introducing one or more amino acid substitutions, the spacer sequence-independent collateral cleavage activity (“collateral activity”, “off-target cleavage activity”) of a reference Cas13f polypeptide (wild type, “WT”, SEQ ID NO: 1) may be substantially decreased (“cleavage activity”, “on-target cleavage activity”) of the reference Cas13f polypeptide.

### Designs and Constructions:

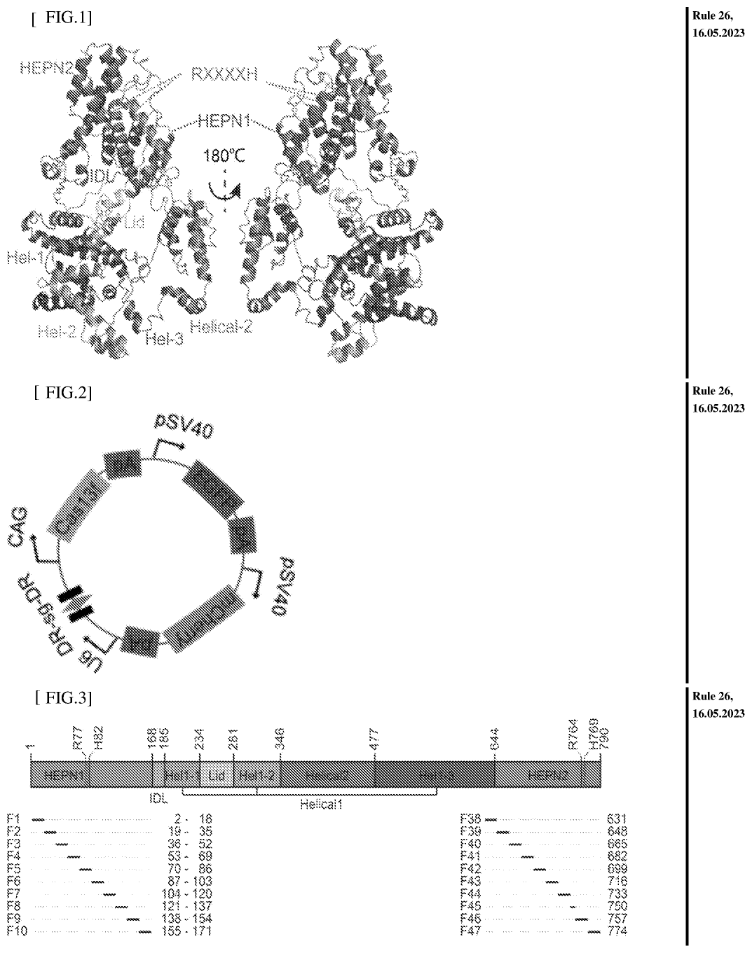

A publicly available online tool TASSER was used to predict the 3D structure of the reference Cas13f polypeptide (SEQ ID NO: 1), and the predicted structure was visualized with PyMOL as shown in FIG. 1 to predict the position of the various structural domains in 3D.

A one-plasmid mammalian dual-fluorescence reporter system as shown in FIG. 2 was constructed for detection of the collateral activities of Cas13f mutants designed based on the reference Cas13f polypeptide.

The plasmid comprised (1) a Cas13f mutant coding sequence flanked by both 5′ and 3′ SV40 NLS (SEQ ID NO: 5) coding sequences under the regulation of a CAG promoter and a poly A sequence, (2) a EGFP green fluorescent reporter gene (with its RNA transcript as an RNA target for cleavage activity of the Cas13f mutants) under the regulation of a SV40 promoter and a poly A sequence, (3) a mCherry red fluorescent reporter gene (with its RNA transcript as an RNA target for collateral activity of the Cas13f mutants) under the regulation of a SV40 promoter and a poly A sequence, and (4) a sequence encoding a EGFP-targeting guide RNA (SEQ ID NO: 15) consisting of 5′-DR sequence (SEQ ID NO: 2)-EGFP-targeting spacer sequence (SEQ ID NO: 6)-DR sequence (SEQ ID NO: 2)-3′ under the regulation of a U6 promoter.

The HEPN1, HEPN2, IDL, and Hell-3 domains of the reference Cas13f polypeptide were chosen for generating a Cas13f mutagenesis library. 20 small segments were selected over those domains (F1-F10 and F38-F47, FIG. 3), each with 17 residues except for F45V1 and F45V2 (each with 9 residues).

For designing Cas13f mutants, the non-Ala (A) residues of each segment, if present, were substituted with Ala (A) residues in several versions, and the Ala (A) residues of each segment, if present, were substituted with Val (V) residues in several versions. For example, for F1 segment, F1V1-F1V4 mutants were designed. About 4-5 total mutations were introduced into each segment in each version to generate a Cas13f mutant. The Cas13f mutants so generated and the amino acid sequences of the mutated segment are provided in Table 1 below, and the other part of each of the Cas13f mutants is the same as the reference Cas13f polypeptide of SEQ ID NO: 1.

TABLE 1
Design of Cas13f mutants

| Mutant | Amino Acid Sequence | Mutant | Amino Acid Sequence | Mutant | Amino Acid Sequence |
|---|---|---|---|---|---|
| F1V1 | ANIELAAEEAAFAFNQA | F16V4 | KDIFVAEEAFQGNSYFA | F32V2 | KHGIEAAALRITIDIAK |
| F1V2 | NGAEAKKEEAAFYFAAA | F17V1 | INAHAAVIAEDELAELC | F32V3 | KAGAENLNARATIDINK |
| F1V3 | NGIALKKAEAAAYANQA | F17V2 | LAGHKGAIGEAEAKELC | F32V4 | KHGIANLNLAITADANK |
| F1V4 | NGIELKKEAVVFYFNQV | F17V3 | ANGAKGVIGEDELKEAA | F33V1 | ARAAVLARIAIPRAFVA |
| F2V1 | ELALAATEANIFAAERR | F17V4 | INGHKGVAGADALKALC | F33V2 | SRKAAANRAAIPRGFAK |
| F2V2 | LANAKAAEDAIFDKERR | F18V1 | AAFLIANQAANAVEARI | F33V3 | SRKVVLNRIAAARGAVK |
| F2V3 | ALNLKAIADNAADKERR | F18V2 | YAFLIGAADAAKAEGRI | F33V4 | SAKAVLNAIVIPAGFVK |
| F2V4 | ELNLKVIEDNIEDKAAA | F18V3 | YAAAAGNQDANKVEGRA | F34V1 | RHILGWQEAEAVAAAIR |
| F3V1 | AALLAAPQILAAMENFI | F18V4 | YVFLIGNQDVNKVAGAI | F34V2 | RHIAAWAESEKASKKIR |
| F3V2 | KTAANNPAILAKMEAFI | F19V1 | AQFLEAFRAANAVQQVA | F34V3 | RAALGWQASEKVSKKAR |
| F3V3 | KTLLNNPQAAAKAENFA | F19V2 | TAFLEKERNAASAQQAK | F34V4 | AHILGAQESAKVSKKIA |
| F3V4 | KTLLNNAQILVKMANAI | F19V3 | TQAAEKFRNANSVAAVK | F35V1 | EAECEILLAABAEELAA |
| F4V1 | FNFRAVAANAAAEIDCL | F19V4 | TQFLAKAANVNSVQQVK | F35V2 | EAEAEIAASKEYEEASK |
| F4V2 | FAFRDATKAAKGELACL | F20V1 | AAEMLAPEAFPANAEAE | F35V3 | AAACAALLSKEYEELSK |
| F4V3 | ANFRDVTKNAKGEADAA | F20V2 | DDEAAKPEYAPAAYFAE | F35V4 | EVECEILLSKAYAALSK |
| F4V4 | FNAADVTKNVKGAIDCL | F20V3 | DDAMLKPAYFPANYAAA | F36V1 | QFFQAADYDAMARINAL |
| F5V1 | ALALRELRNFYSHAAHA | F20V4 | DDEMLKAEYFAVNYFVE | F36V2 | QFFQSKAAAKMTRIAGL |
| F5V2 | LAKAREARNFYSHYVAK | F21V1 | AAVARIADRVLNRLNAA | F36V3 | AFFASKDYDKATRINGA |
| F5V3 | LLKLAALRNFYSHYVHK | F21V2 | SGAGRIKARVLARLAKA | F36V4 | QAAQSKDYDKMTAANGL |
| F6V1 | RDVRELAAAEAPILEAY | F21V3 | SGVGRAKDRAANRANKA | F37V1 | AEANALIALMAVALMAQ |
| F6V2 | RAAREASKGEKPILEKA | F21V4 | SGVGAIKDAVLNALNKV | F37V2 | YEKAKAIALMAAYLMGA |
| F6V3 | RDVRALSKGAKPAAEKY | F22V1 | LASNAAAAGEIIAYDAM | F37V3 | YEKNKLIAAAAVYAAGQ |
| F6V4 | ADVAELSKGEKAILAKY | F22V2 | IKANKAKKAEIIAAAKM | F37V4 | YAKNKLAVLMVVYLMGQ |
| F7V1 | YQFALEAAAAENVALEI | F22V3 | AKSAKAKKGEALAYDKA | F38V1 | LRILFAEHAALDDIAAT |
| F7V2 | AAFAIESTGSEAAKLEI | F22V4 | IKSNKVKKGALAVYDKM | F38V2 | ARILFKEHTKLAAITKA |
| F7V3 | YQAAAESTGSENVKAEA | F23V1 | REVMAFIAAALPVAEAL | F38V3 | LRAAFKEATKADDITKT |
| F7V4 | YQFVIASTGSANVKLAI | F23V2 | REAMAFINNSAPADEKA | F38V4 | LAILAKAHTKLDDATKT |
| F8V1 | IAAAWLAAAAALFFLC | F23V3 | RAVAAAANNSLPVDEKL | F39V1 | TVDFAIADAVTVAIPFA |
| F8V2 | IENDAWAADAGVAFFAA | F23V4 | AEVMVFINNSLAVDAKL | F39V2 | AVAFKISAKVAVKIPFS |
| F8V3 | AANDAWLADAGVLAALC | F24V1 | APAAYARYLAMVRFWDR | F39V3 | TADFKASDKATAKIPFS |
| F8V4 | IENDVALVDVGVLFFLC | F24V2 | KPKDAKRALGMARFWAR | F39V4 | TVDAKISDKVTVKAAAS |
| F9V1 | IFLAAAQANALLAGISG | F24V3 | KAKDYKRYAGAVRAWDR | F40V1 | NYPALVYAMAAAYVDNI |
| F9V2 | IFLKKSQAAKLISAIAA | F24V4 | KPKDYKAYLGMVAFADA | F40V2 | NAPSLVATMSSKAVANI |
| F9V3 | AFAKKSAANKAISGISG | F25V1 | EADNIAREFETAEWAAY | F40V3 | AYPSLAYTMSSKYADAI |
| F9V4 | IALKKSQVNKLASGASG | F25V2 | EKAAIKREFEAKEWSKA | F40V4 | NYASAVYTASSKYVDNA |
| F10V1 | FARNADAAQPRRNLFAY | F25V3 | AKDNAKRAAETKEWSKY | F41V1 | GNYGFANADADAPILGA |
| F10V2 | FKRADATGQPRRALFTA | F25V4 | EKDNIKAEFATKAASKY | F41V2 | ANYAFSNKAKDKPILAK |
| F10V3 | AKRNDDTGAPRRNAATY | F26V1 | LPANFWAAANLERVAAL | F41V3 | GAAGFSAKDKAKPILGK |
| F10V4 | FKANDDTGQAAANLFTY | F26V2 | APSAFWTAKALERAYGL | F41V4 | GNYGASNKDKDKAAAGK |
| F11V1 | FAIREAAAVVPEMQAHF | F26V3 | LPSNAWTAKNAARVYGA | F42V1 | LAAIEAQRMEFIAEVLA |
| F11V2 | FSIREGYKAAPEMAKAF | F26V4 | LASNFATVKNLEAVYGL | F42V2 | IDVIEKARAEFIKEAAG |
| F11V3 | ASAREGYKVVPEAQKHA | F27V1 | AREAAAELFNALAAAVE | F42V3 | ADVAEKQRMEAAKEVLG |
| F11V4 | FSIAAGYKVVAAMQKHF | F27V2 | AREKNAEAFAKAKADAE | F42V4 | IDVIAKQAMAFIKAVLG |
| F12V1 | LLFALVNHLANQAAAIE | F27V3 | ARAKNAALANKLKADVA | F43V1 | FEAYLFDDALIDAAAFA |
| F12V2 | LLFSLAAHLSAADDYIE | F27V4 | VAEKNVELFNKLKVDVE | F43V2 | FEKALFAAKIIAKSKFA |
| F12V3 | AAFSAVNHASNQDDYTE | F28V1 | AMAERELEAYQAINDAA | F43V3 | AEKYAFDDKAADKSKFA |
| F12V4 | LLASLVNALSNQDDYAA | F28V2 | KMDERELEKAAKIAAAK | F43V4 | FAKYLADDKIIDKSKAV |
| F13V1 | AAHQPAAIAEALFFHRI | F28V3 | KADAREAEKYQKANDAK | F44V1 | AAAAHIAFAEIAEELVE |
| F13V2 | KAAAPYDIGEGAFFARI | F28V4 | KMDEAALAKYQKINDVK | F44V2 | DTATAASFAEIVEEAAE |
| F13V3 | KAHQPYDAGEGLAAHRA | F29V1 | ALANLRRLAAAFAVAWE | F44V3 | DTATHISAAAAVAELVE |
| F13V4 | KVHQAYDIGAGLFFHAI | F29V2 | DAAAARRLASDFGAKWE | F44V4 | DTVTHISFVEIVEALVA |
| F14V1 | AAAFLNIAAILRNMAFY | F29V3 | DLVNLRRAASDAGVKWA | F45V1 | AAWAADRLA |
| F14V2 | ASTFAAISGILRAMKFA | F29V4 | DLANLAALVSDFGVKAE | F45V2 | KGADKAAAT |
| F14V3 | ASTFLNASGAARNAKFY | F30V1 | EADWDEYAAQIAAQITD | F46V1 | LAALAAARNKALHAEIL |
| F14V4 | VSTALNISGILANMKAY | F30V2 | EKAWAEYSGQIKKQLAA | F46V2 | ATKAKDARNKALHGEAA |
| F15V1 | AYQAARLVEQRAELARE | F30V3 | EKDWDEASGAAKKAITD | F46V3 | LTKLKDVRNKALHGAIL |
| F15V2 | TAASKRLAEARGELKRE | F30V4 | AKDADAYSGQIKKQATD | F47V1 | TGTAFDETAALINELAA |
| F15V3 | TYQSKRAVAQRGAAKRE | F31V1 | AQALTIMAQRITAGLAA | F47V2 | AAASFDEAKSLINELKK |
| F15V4 | TYQSKALVEQAGELKAA | F31V2 | SAKLAIMKQRIAAALKK | F47V3 | TGTSFAETKSAIAEAKK |
| F16V1 | AAIFAWEEPFQANAAFE | F31V3 | SQKATIAKARITAGAKK | F47V4 | TGTSADATKSLANALKK |
| F16V2 | KDAAAWEEPFAGASYFE | F31V4 | SQKLTAMKQAATVGLKK | | |
| F16V3 | KDIFAWAAPAQGNSYAE | F32V1 | AHAIENLNLRIAIAINA | | |

### Transfection and Detection:

HEK293T cells were cultured in 24-well tissue culture plates according to standard methods for 12 hours, before the plasmid was transfected into the cells using standard polyethyleneimine (PEI) transfection. The transfected cells were then cultured at 37° C. under 5% CO₂ for about 48 hours. Then the cultured cells were analyzed by flow cytometry.

Dead Cas13f (“dCas13f”, “dead”) (Cas13f mutant with R77A, H82A, R764A, and H769A mutations in HEPN domains of the reference Cas13f polypeptide of SEQ ID NO: 1) with no cleavage and collateral activities was used as a negative control (“NT”).

The cleavage activity of each tested Cas13f mutant was inversely correlated to the percentage proportion of EGFP positive cells (% EGFP⁺). The lower the % EGFP⁺ is, the higher the cleavage activity would be. Therefore, the cleavage activity of the Cas13f mutant relative to the reference Cas13f polypeptide (“WT”) of SEQ ID NO: 1 is shown as the reciprocal of % EGFP⁺ of Cas13f mutant relative to % EGFP⁺ of WT.

$$Cleavage activity relative to WT = 1 / ( \% EGF {P}^{+} of Cas 13 f mutant / \% {EGFP}^{+} of WT ) ) .$$

The collateral activity of each tested Cas13f mutant was inversely correlated to the percentage proportion of mCherry positive cells (% mCherry⁺). The higher the % mCherry⁺ is, the lower the collateral activity would be. Therefore, the collateral activity of the Cas13f mutant relative to the reference Cas13f polypeptide (“WT”) of SEQ ID NO: 1 is shown as the reciprocal of % mCherry+ of Cas13f mutant relative to % mCherry⁺ of WT.

$$Collateral activity relative to WT = 1 / ( \% {mCherry}^{+} of Cas 13 f mutant / \% {mCherry}^{+} of WT ) ) .$$

### Results

The flow cytometry results (Table 2) show the cleavage and collateral activities of the Cas13f mutants.

TABLE 2
Averaged cleavage and collateral activities
of the Cas13f mutants in Table 1 (n = 3)

| | | Collateral | | Cleavage |
|---|---|---|---|---|
| | | Activity | | Activity |
| | | Relative to | | Relative to |
| Mutant | % mCherry⁺ | WT | % EGFP⁺ | WT |
| F40V2 | 98.57% | 53.55% | 27.11% | 34.82% |
| F3V2 | 97.49% | 54.14% | 49.88% | 18.93% |
| F4V2 | 94.68% | 55.75% | 90.54% | 10.43% |
| F44V3 | 94.20% | 56.03% | 88.56% | 10.66% |
| F5V1 | 93.85% | 56.24% | 53.79% | 17.55% |
| F39V3 | 92.12% | 57.29% | 35.47% | 26.61% |
| F39V4 | 91.68% | 57.57% | 42.72% | 22.10% |
| F43V4 | 91.33% | 57.79% | 81.46% | 11.59% |
| F4V4 | 90.46% | 58.35% | 74.16% | 12.73% |
| F7V2 | 89.15% | 59.20% | 19.56% | 48.26% |
| F46V2 | 88.99% | 59.31% | 87.97% | 10.73% |
| F42V3 | 88.21% | 59.83% | 87.02% | 10.85% |
| F44V3 | 88.13% | 59.89% | 59.60% | 15.84% |
| F41V4 | 87.26% | 60.49% | 34.35% | 27.48% |
| F9V1 | 86.56% | 60.98% | 45.49% | 20.75% |
| F43V3 | 85.38% | 61.82% | 73.31% | 12.88% |
| F40V4 | 85.24% | 61.92% | 16.98% | 55.59% |
| F44V2 | 85.19% | 61.96% | 27.28% | 34.60% |
| F47V1 | 85.00% | 62.09% | 49.65% | 19.01% |
| F2V1 | 84.39% | 62.54% | 62.76% | 15.04% |
| F7V4 | 83.93% | 62.89% | 69.18% | 13.65% |
| F10V3 | 83.60% | 63.13% | 71.23% | 13.25% |
| F47V4 | 83.56% | 63.16% | 70.86% | 13.32% |
| F6V1 | 83.09% | 63.52% | 28.58% | 33.03% |
| F2V2 | 82.66% | 63.85% | 43.40% | 21.75% |
| F8V2 | 82.28% | 64.15% | 78.36% | 12.05% |
| F10V4 | 82.15% | 64.25% | 9.29% | 101.61% |
| F8V3 | 81.80% | 64.52% | 81.01% | 11.65% |
| F8V1 | 81.71% | 64.59% | 79.74% | 11.84% |
| F10V1 | 81.31% | 64.91% | 21.23% | 44.47% |
| F41V3 | 74.46% | 70.88% | 8.86% | 106.55% |
| F3V3 | 72.97% | 72.33% | 25.25% | 37.39% |
| F9V3 | 69.49% | 75.95% | 71.16% | 13.27% |
| F6V2 | 69.13% | 76.35% | 36.36% | 25.96% |
| F7V3 | 68.61% | 76.93% | 41.22% | 22.90% |
| F42V2 | 68.10% | 77.50% | 51.06% | 18.49% |
| F4V1 | 67.83% | 77.81% | 34.02% | 27.75% |
| F42V4 | 67.18% | 78.57% | 20.16% | 46.83% |
| F9V4 | 66.77% | 79.05% | 63.70% | 14.82% |
| F40V1 | 64.40% | 81.96% | 8.56% | 110.28% |
| F2V4 | 62.68% | 84.21% | 21.91% | 43.09% |
| F6V4 | 62.62% | 84.29% | 12.83% | 73.58% |
| F3V4 | 59.09% | 89.32% | 11.12% | 84.89% |
| F38V4 | 58.60% | 90.07% | 9.04% | 104.42% |
| F3V1 | 57.84% | 91.25% | 19.15% | 49.30% |
| F43V1 | 55.08% | 95.82% | 19.99% | 47.22% |
| F5V3 | 54.05% | 97.65% | 16.88% | 55.92% |
| F5V2 | 53.52% | 98.62% | 12.81% | 73.69% |
| F7V1 | 53.15% | 99.30% | 9.60% | 98.33% |
| F41V1 | 52.81% | 99.94% | 9.63% | 98.03% |
| WT | 52.78% | 100.00% | 9.44% | 100.00% |
| F9V2 | 49.51% | 106.60% | 10.51% | 89.82% |
| F44V1 | 49.36% | 106.93% | 5.85% | 161.37% |
| F45V1 | 49.07% | 107.56% | 12.29% | 76.81% |
| F7V4 | 47.94% | 110.10% | 27.48% | 34.35% |
| F39V1 | 47.31% | 111.56% | 7.36% | 128.26% |
| F39V2 | 46.39% | 113.77% | 3.86% | 244.56% |
| F2V3 | 44.29% | 119.17% | 14.45% | 65.33% |
| F47V1 | 43.31% | 121.87% | 6.02% | 156.81% |
| F46V1 | 41.39% | 127.52% | 4.77% | 197.90% |
| F47V3 | 37.90% | 139.26% | 6.07% | 155.52% |
| F41V2 | 35.67% | 147.97% | 6.44% | 146.58% |
| F4V3 | 34.46% | 153.16% | 7.75% | 121.81% |
| F6V3 | 34.26% | 154.06% | 8.29% | 113.87% |
| F38V1 | 32.61% | 161.85% | 3.81% | 247.77% |
| F10V2 | 31.65% | 166.76% | 4.70% | 200.85% |
| F8V4 | 31.62% | 166.92% | 4.94% | 191.09% |
| F38V3 | 30.78% | 171.47% | 5.26% | 179.47% |
| F45V2 | 30.45% | 173.33% | 4.59% | 205.66% |
| F47V2 | 29.73% | 177.53% | 3.47% | 272.05% |
| F43V2 | 29.09% | 181.44% | 2.93% | 322.18% |
| F44V4 | 28.71% | 183.84% | 2.62% | 360.31% |
| F40V3 | 26.44% | 199.62% | 3.41% | 276.83% |
| F42V1 | 23.98% | 220.10% | 3.06% | 308.50% |
| F38V2 | 20.31% | 259.87% | 3.50% | 269.71% |
| F46V3 | 20.17% | 261.68% | 1.99% | 474.37% |
| NT | 100.00% | — | 100.00% | — |

The Cas13f mutants are arranged in Table 2 in an order of low-to-high collateral activity relative to WT. It was noted that among those Cas13f mutans having less than 70% collateral activity relative to WT (highlighted in grey), the Cas13f mutants F7V2, F10V1, F10V4, F40V2, and F40V4 have the top 5 high cleavage activity relative to WT (highlighted in grey).

A second round of mutagenesis study in or nearby the selected regions of the Cas13f mutants was conducted by generating a number of additional Cas13f mutants with a single or multiple (e.g., double, triple, or quadruple) mutations. The Cas13f mutants so generated and the amino acid sequences of the mutated segment are provided in Table 3 below, and the other part of each of the Cas13f mutants is the same as the reference Cas13f polypeptide of SEQ ID NO: 1. Their cleavage and collateral activities are listed in Table 4 below.

TABLE 3
Design of Cas13f mutants

| Mutant | Amino Acid Sequence | Mutant | Amino Acid Sequence | Mutant | Amino Acid Sequence |
|---|---|---|---|---|---|
| F10S1 | AKRNDDTGQPRRNLFTY | F40S7 | NYPSLVATMSSKYVDNI | F10S33 | FARNADTGQPRRNLFTY |
| F10S2 | FARNDDTGQPRRNLFTY | F40S8 | NYPSLVYAMSSKYVDNI | F10S34 | FARNDDAGQPRRNLFTY |
| F10S3 | FKANDDTGQPRRNLFTY | F40S9 | NYPSLVYTASSKYVDNI | F10S35 | FARNDDTAQPRRNLFTY |
| F10S4 | FKRADDTGQPRRNLFTY | F40S10 | NYPSLVYTMASKYVDNI | F10S36 | FARNDDTGQPRRNLFAY |
| F10S5 | FKRNADTGQPRRNLFTY | F40S11 | NYPSLVYTMSAKYVDNI | F10S37 | FKRNADAGQPRRNLFTY |
| F1086 | FKRNDATGQPRRNLFTY | F40S12 | NYPSLVYTMSSAYVDNI | F10S38 | FKRNADTAQPRRNLFTY |
| F10S7 | FKRNDDAGQPRRNLFTY | F40813 | NYPSLVYTMSSKAVDNI | F10S39 | FKRNADTGQPRRNLFAY |
| F10S8 | FKRNDDTAQPRRNLFTY | F40S14 | NYPSLVYTMSSKYADNI | F10S40 | FKRNDDAAQPRRNLFTY |
| F10S9 | FKRNDDTGAPRRNLFTY | F40S15 | NYPSLVYTMSSKYVANI | F10S41 | FKRNDDAGQPRRNLFAY |
| F10S10 | FKRNDDTGQARRNLFTY | F40S16 | NYPSLVYTMSSKYVDAI | F10S42 | FKRNDDTAQPRRNLFAY |
| F10S11 | FKRNDDTGQPARNLFTY | F40S17 | NYPSLVYTMSSKYVDNA | F10S43 | FKANDDTGQAARNLFTY |
| F10S12 | FKRNDDTGQPRANLFTY | F46S1 | ATKLKDARNKALHGEIL | F10S44 | FKANDDTGQARANLFTY |
| F10S13 | FKRNDDTGQPRRALFTY | F46S2 | LAKLKDARNKALHGEIL | F10S45 | FKANDDTGQPAANLFTY |
| F10S14 | FKRNDDTGQPRRNAFTY | F46S3 | LTALKDARNKALHGEIL | F10S46 | FKRNDDTGQAAANLFTY |
| F10S15 | FKRNDDTGQPRRNLATY | F46S4 | LTKAKDARNKALHGEIL | F10S47 | FKANDDTGQARRNLFTY |
| F10S16 | FKRNDDTGQPRRNLFAY | F46S5 | LTKLADARNKALHGEIL | F10S48 | FKANDDTGQPARNLFTY |
| F10S17 | FKRNDDTGQPRRNLFTA | F4686 | LTKLKAARNKALHGEIL | F10S49 | FKANDDTGQPRANLFTY |
| F38S1 | ARILFKEHTKLDDITKT | F46S7 | LTKLKDVRNKALHGEIL | F10S50 | FKRNDDTGQAARNLFTY |
| F38S2 | LAILFKEHTKLDDITKT | F46S10 | LTKLKDARNAALHGEIL | F10S51 | FKRNDDTGQARANLFTY |
| F38S3 | LRALFKEHTKLDDITKT | F46S11 | LTKLKDARNKVLHGEIL | F10S52 | FKRNDDTGQPAANLFTY |
| F38S4 | LRIAFKEHTKLDDITKT | F46S12 | LTKLKDARNKAAHGEIL | F40S18 | NAPSLVATMSSKAVDNI |
| F38S5 | LRILAKEHTKLDDITKT | F46S14 | LTKLKDARNKALHAEIL | F40S19 | NAPSLVATMSSKYVANI |
| F38S6 | LRILFAEHTKLDDITKT | F46S15 | LTKLKDARNKALHGAIL | F40S20 | NAPSLVYTMSSKAVANI |
| F38S7 | LRILFKAHTKLDDITKT | F46S16 | LTKLKDARNKALHGEAL | F40S21 | NYPSLVATMSSKAVANI |
| F3888 | LRILFKEATKLDDITKT | F46S17 | LTKLKDARNKALHGEIA | F40S22 | NAPSLVATMSSKYVDNI |
| F38S9 | LRILFKEHAKLDDITKT | F10S18 | FARNADAAQPRRNLFTY | F40S23 | NAPSLVYTMSSKAVDNI |
| F38S10 | LRILFKEHTALDDITKT | F10S19 | FARNADAGQPRRNLFAY | F40S24 | NAPSLVYTMSSKYVANI |
| F38S11 | LRILFKEHTKADDITKT | F10S20 | FARNADTAQPRRNLFAY | F40S25 | NYPSLVATMSSKAVDNI |
| F38S12 | LRILFKEHTKLADITKT | F10S21 | FARNDDAAQPRRNLFAY | F40S26 | NYPSLVATMSSKYVANI |
| F38S13 | LRILFKEHTKLDAITKT | F10S22 | FKRNADAAQPRRNLFAY | F40S27 | NYPSLVYTMSSKAVANI |
| F38S14 | LRILFKEHTKLDDATKT | F10S23 | FARNADAGQPRRNLFTY | F40S28 | NYASAVYTASSKYVDNI |
| F38S15 | LRILFKEHTKLDDIAKT | F10S24 | FARNADTAQPRRNLFTY | F40S29 | NYASAVYTMSSKYVDNA |
| F38S16 | LRILFKEHTKLDDITAT | F10S25 | FARNADTGQPRRNLFAY | F40S30 | NYASLVYTASSKYVDNA |
| F38S17 | LRILFKEHTKLDDITKA | F10S26 | FARNDDAAQPRRNLFTY | F40S31 | NYPSAVYTASSKYVDNA |
| F40S1 | AYPSLVYTMSSKYVDNI | F10S27 | FARNDDAGQPRRNLFAY | F40S32 | NYASAVYTMSSKYVDNI |
| F40S2 | NAPSLVYTMSSKYVDNI | F10S28 | FARNDDTAQPRRNLFAY | F40S33 | NYASLVYTASSKYVDNI |
| F40S3 | NYASLVYTMSSKYVDNI | F10S29 | FKRNADAAQPRRNLFTY | F40S34 | NYASLVYTMSSKYVDNA |
| F40S4 | NYPALVYTMSSKYVDNI | F10S30 | FKRNADAGQPRRNLFAY | F40S35 | NYPSAVYTASSKYVDNI |
| F40S5 | NYPSAVYTMSSKYVDNI | F10S31 | FKRNADTAQPRRNLFAY | F40S36 | NYPSAVYTMSSKYVDNA |
| F40S6 | NYPSLAYTMSSKYVDNI | F10S32 | FKRNDDAAQPRRNLFAY | F40S37 | NYPSLVYTASSKYVDNA |

TABLE 4
Averaged cleavage and collateral activities
of the Cas13f mutants in Table 3 (n = 3)

| | | Collateral | | Cleavage |
|---|---|---|---|---|
| | | Activity | | Activity |
| | | Relative to | | Relative to |
| Mutant | % mCherry⁺ | WT | % EGFP⁺ | WT |
| F40V2 | 97.30% | 33.72% | 35.12% | 12.24% |
| F40S21 | 90.66% | 36.19% | 26.23% | 16.39% |
| F46S3 | 90.32% | 36.33% | 86.15% | 4.99% |
| F10S45 | 90.27% | 36.35% | 20.48% | 21.00% |
| F10S25 | 89.51% | 36.66% | 28.09% | 15.31% |
| F40S26 | 87.07% | 37.68% | 16.33% | 26.33% |
| F10S49 | 86.39% | 37.98% | 13.26% | 32.43% |
| F40S23 | 86.38% | 37.98% | 14.48% | 29.70% |
| F40S31 | 85.97% | 38.16% | 29.81% | 14.42% |
| F10S48 | 84.92% | 38.64% | 10.15% | 42.36% |
| F40S36 | 84.91% | 38.64% | 21.74% | 19.78% |
| F10S33 | 83.36% | 39.36% | 15.15% | 28.38% |
| F40S27 | 81.81% | 40.11% | 8.77% | 49.03% |
| F10S35 | 81.50% | 40.26% | 11.26% | 38.19% |
| F40S22 | 81.19% | 40.41% | 13.85% | 31.05% |
| F10S36 | 81.09% | 40.46% | 21.22% | 20.26% |
| F10S26 | 79.52% | 41.26% | 14.58% | 29.49% |
| F10S19 | 79.44% | 41.30% | 27.44% | 15.67% |
| F10S46 | 79.02% | 41.52% | 14.62% | 29.41% |
| F10S34 | 78.65% | 41.72% | 10.81% | 39.78% |
| F10S27 | 78.63% | 41.73% | 20.90% | 20.57% |
| F10S24 | 77.93% | 42.10% | 13.93% | 30.87% |
| F10S21 | 76.93% | 42.65% | 23.26% | 18.49% |
| F10V1 | 76.12% | 43.10% | 36.23% | 11.87% |
| F46S2 | 75.80% | 43.28% | 83.89% | 5.13% |
| F40V4 | 73.51% | 44.63% | 16.53% | 26.01% |
| F10S28 | 73.15% | 44.85% | 21.12% | 20.36% |
| F10S23 | 73.04% | 44.92% | 14.92% | 28.82% |
| F10S50 | 72.44% | 45.29% | 9.16% | 46.94% |
| F10S18 | 70.99% | 46.22% | 13.04% | 32.98% |
| F10S44 | 70.46% | 46.57% | 9.39% | 45.79% |
| F10S52 | 69.55% | 47.17% | 19.42% | 22.14% |
| F10S43 | 69.47% | 47.23% | 5.15% | 83.50% |
| F10V4 | 69.16% | 47.44% | 10.36% | 41.51% |
| F40S25 | 68.11% | 48.17% | 33.03% | 13.02% |
| F40S37 | 67.07% | 48.92% | 12.95% | 33.20% |
| F10S20 | 66.74% | 49.16% | 25.15% | 17.10% |
| F10S11 | 65.01% | 50.47% | 9.00% | 47.78% |
| F10S51 | 64.41% | 50.94% | 9.48% | 45.36% |
| F10S12 | 64.24% | 51.07% | 7.51% | 57.26% |
| F40S30 | 64.16% | 51.14% | 16.69% | 25.76% |
| F10S43 | 63.40% | 51.75% | 5.93% | 72.51% |
| F40S7 | 63.27% | 51.86% | 7.29% | 58.98% |
| F10S2 | 60.95% | 53.83% | 8.08% | 53.22% |
| F40S28 | 59.78% | 54.88% | 6.65% | 64.66% |
| F40S35 | 57.38% | 57.18% | 8.02% | 53.62% |
| F40S24 | 56.52% | 58.05% | 4.14% | 103.86% |
| F10S31 | 56.32% | 58.26% | 15.32% | 28.07% |
| F10S47 | 56.27% | 58.31% | 5.79% | 74.27% |
| F40S9 | 50.53% | 64.93% | 8.76% | 49.09% |
| F40S29 | 50.32% | 65.20% | 10.80% | 39.81% |
| F40S11 | 50.24% | 65.31% | 9.66% | 44.51% |
| F40S17 | 49.58% | 66.18% | 12.54% | 34.29% |
| F10S39 | 49.52% | 66.26% | 16.10% | 26.71% |
| F40S12 | 48.81% | 67.22% | 11.61% | 37.04% |
| F40S13 | 48.52% | 67.62% | 18.70% | 22.99% |
| F10S1 | 47.87% | 68.54% | 9.32% | 46.14% |
| F46S11 | 47.44% | 69.16% | 8.00% | 53.75% |
| F46V1 | 46.65% | 70.33% | 10.38% | 41.43% |
| F40S32 | 46.55% | 70.48% | 6.65% | 64.66% |
| F10S42 | 45.67% | 71.84% | 9.60% | 44.79% |
| F40S14 | 44.60% | 73.57% | 12.38% | 34.73% |
| F10S38 | 44.43% | 73.85% | 9.31% | 46.19% |
| F10S22 | 44.22% | 74.20% | 12.72% | 33.81% |
| F46S16 | 43.54% | 75.36% | 5.55% | 77.48% |
| F10S15 | 42.10% | 77.93% | 11.36% | 37.85% |
| F10S30 | 41.86% | 78.38% | 12.43% | 34.59% |
| F10S16 | 41.00% | 80.02% | 11.98% | 35.89% |
| F40S19 | 39.01% | 84.11% | 14.82% | 29.01% |
| F40S10 | 38.43% | 85.38% | 4.55% | 94.51% |
| F40S33 | 37.23% | 88.13% | 5.87% | 73.25% |
| F40S20 | 36.77% | 89.23% | 20.81% | 20.66% |
| F10S41 | 36.42% | 90.09% | 7.83% | 54.92% |
| F10S29 | 36.34% | 90.29% | 5.08% | 84.65% |
| F46S5 | 35.62% | 92.11% | 3.54% | 121.47% |
| WT | 32.81% | 100.00% | 4.30% | 100.00% |
| F10S37 | 32.27% | 101.67% | 4.32% | 99.54% |
| F40S15 | 32.20% | 101.89% | 10.02% | 42.91% |
| F10S40 | 32.02% | 102.47% | 2.82% | 152.48% |
| F38S3 | 31.50% | 104.16% | 4.53% | 94.92% |
| F10S32 | 31.35% | 104.66% | 6.19% | 69.47% |
| F40S34 | 30.51% | 107.54% | 4.45% | 96.63% |
| F10S17 | 29.73% | 110.36% | 6.75% | 63.70% |
| F40S18 | 29.38% | 111.67% | 20.85% | 20.62% |
| F10S13 | 29.06% | 112.90% | 3.52% | 122.16% |
| F10S8 | 28.72% | 114.24% | 2.61% | 164.75% |
| F46S12 | 28.56% | 114.88% | 6.74% | 63.80% |
| F10S3 | 28.01% | 117.14% | 2.46% | 174.80% |
| F10S14 | 27.91% | 117.56% | 3.36% | 127.98% |
| F46S17 | 27.08% | 121.16% | 3.32% | 129.52% |
| F40S16 | 25.60% | 128.16% | 9.79% | 43.92% |
| F10S7 | 24.93% | 131.61% | 2.43% | 176.95% |
| F38S13 | 24.62% | 133.27% | 2.72% | 158.09% |
| F46S2 | 24.44% | 134.25% | 2.58% | 166.67% |
| F10S10 | 23.55% | 139.32% | 1.97% | 218.27% |
| F40S1 | 23.02% | 142.53% | 2.57% | 167.32% |
| F40S8 | 22.95% | 142.96% | 2.73% | 157.51% |
| F40S5 | 22.66% | 144.79% | 3.32% | 129.52% |
| F46S4 | 22.22% | 147.66% | 5.19% | 82.85% |
| F38V2 | 22.17% | 147.99% | 3.30% | 130.30% |
| F46S7 | 21.88% | 149.95% | 2.41% | 178.42% |
| F38S1 | 21.47% | 152.82% | 1.91% | 225.13% |
| F40S2 | 21.38% | 153.46% | 1.89% | 227.51% |
| F46S1 | 21.37% | 153.53% | 4.13% | 104.12% |
| F46S10 | 21.36% | 153.60% | 3.09% | 139.16% |
| F38S5 | 21.33% | 153.82% | 2.85% | 150.88% |
| F38S2 | 21.31% | 153.97% | 2.99% | 143.81% |
| F38S6 | 20.66% | 158.81% | 2.34% | 183.76% |
| F40S6 | 20.38% | 160.99% | 2.41% | 178.42% |
| F38S17 | 20.16% | 162.75% | 2.03% | 211.82% |
| F38S9 | 19.97% | 164.30% | 2.58% | 166.67% |
| F38S13 | 19.83% | 165.46% | 2.78% | 154.68% |
| F38S8 | 19.61% | 167.31% | 2.02% | 212.87% |
| F38S16 | 19.40% | 169.12% | 2.27% | 189.43% |
| F40S3 | 17.89% | 183.40% | 2.01% | 213.93% |
| F38S11 | 17.80% | 184.33% | 2.24% | 191.96% |
| F38S7 | 17.70% | 185.37% | 2.29% | 187.77% |
| F38S15 | 17.22% | 190.53% | 1.78% | 241.57% |
| F46S14 | 16.75% | 195.88% | 2.37% | 181.43% |
| F10S9 | 16.59% | 197.77% | 1.48% | 290.54% |
| F40S4 | 16.33% | 200.92% | 1.98% | 217.17% |
| F38S4 | 16.00% | 205.06% | 2.16% | 199.07% |
| F46S6 | 15.32% | 214.16% | 2.10% | 204.76% |
| F46V3 | 14.19% | 231.22% | 1.34% | 320.90% |
| F38S10 | 13.89% | 236.21% | 1.85% | 232.43% |
| F10S4 | 13.75% | 238.62% | 1.77% | 242.94% |
| F38S12 | 13.53% | 242.50% | 1.70% | 252.94% |
| F10S5 | 13.10% | 250.46% | 2.70% | 159.26% |
| F10S6 | 13.06% | 251.23% | 1.48% | 290.54% |
| F46S15 | 11.06% | 296.65% | 1.31% | 328.24% |
| NT | 100.00% | — | 100.00% | — |

The Cas13f mutants are arranged in Table 4 in an order of low-to-high collateral activity relative to WT. It was noted that among those Cas13f mutans having less than 40% collateral activity relative to WT (highlighted in grey), the Cas13f mutants F10S48, F10S49, F40S23, F10S33, and F40S26 have the top 5 high cleavage activity relative to WT (highlighted in grey).

The Cas13f mutant F40S23 (Cas13f-Y666A, Y677A, SEQ ID NO: 3) is selected for further engineering.

### Example 2 Engineering of Mutant F40S23 for Increased Cleavage Activity

Cas13f mutants had been screened for a low spacer sequence-independent collateral cleavage activity (“collateral activity”, “off-target cleavage activity”) in Example 1. In order to improve the spacer sequence-specific cleavage activity (“cleavage activity”, “on-target cleavage activity”) while maintaining the low collateral activity, one or more mutations as shown in Table 5 identified from Example 1 were further introduced into the Cas13f mutant F40S23.

This Example demonstrates that by introducing one or more amino acid substitutions, the cleavage activity of F40S23 may be substantially increased, and the collateral activity may be substantially maintained or even reduced.

TABLE 5
Available mutations for introduction into mutant F40S23

| Mutation | Corresponding mutant in Example 1 |
|---|---|
| D160A | F10S6 |
| T161A | F10S7 |
| L631A | F38S1 |
| L634A | F38S4 |
| H638A | F38S8 |
| L641A | F38S11 |
| D642A | F38S12 |
| T647A | F38S17 |
| P667A | F40S3 |
| V670A | F40S6 |
| D762A | F46S6 |
| A763V | F46S7 |

### Designs and Constructions:

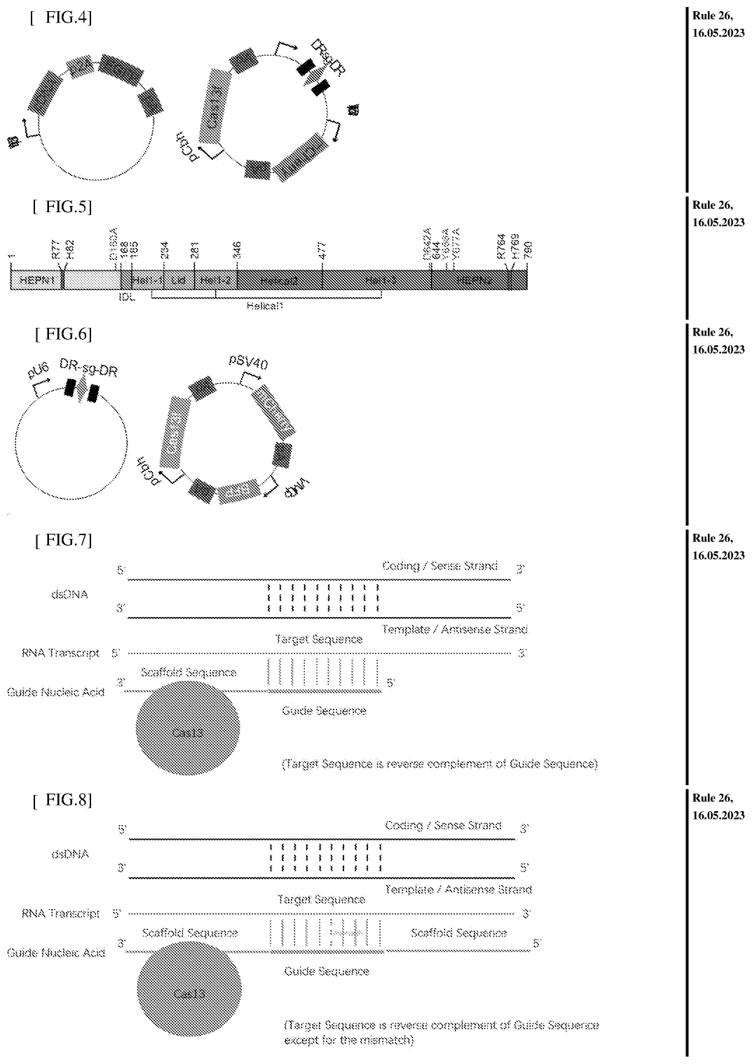

A two-plasmid mammalian fluorescence reporter system (FIG. 4) comprising a reporter plasmid and an expression plasmid was constructed for detection of the cleavage and collateral activities of the Cas13f mutants further engineered based on F40S23.

The reporter plasmid comprised a ATXN2 cDNA coding sequence (with its RNA transcript as an RNA target for cleavage activity of the Cas13f mutants) followed by a p2A (self-cleaving peptide) and an EGFP reporter gene (SEQ ID NO: 7) under the regulation of SV40 promoter and a poly A sequence. EGFP mRNA was transcribed together with the ATXN2 RNA transcript from the reporter plasmid to form a chimeric transcript. When the ATXN2 RNA transcript as a part of the chimeric transcript was cleaved by a Cas13f mutant guided by a ATXN2-targeting gRNA (SEQ ID NO: 16), the EGFR mRNA as another part of the chimeric transcript would also be gradually degraded due to, e.g., overall RNA instability, leading to reduced fluorescent intensity of EGFP (Green).

The expression plasmid comprised (1) a Cas13f mutant coding sequence flanked by both 5′ and 3′ SV40 NLS (SEQ ID NO: 5) coding sequence under the regulation of a Cbh promoter and a poly A sequence, (2) a sequence encoding a ATXN2-targeting gRNA (SEQ ID NO: 16) consisting of 5′-DR sequence (SEQ ID NO: 2)-AXTN2-targeting spacer sequence (SEQ ID NO: 8)-DR sequence (SEQ ID NO: 2)-3′ under the regulation of a U6 promoter and (3) a mCherry reporter gene (with its RNA transcript as an RNA target for collateral activity of the Cas13f mutants) under the regulation of a SV40 promoter and a poly A sequence. In the case that the Cas13f mutant retained a substantial collateral activity, the mCherry RNA transcript may be cleaved, leading to reduced fluorescent intensity of mCherry (Red).

A similar pair of reporter and expression plasmids was constructed in the same way with an RHO cDNA coding sequence followed by a p2A (self-cleaving peptide) and an EGFP reporter gene (SEQ ID NO: 10) and an RHO-targeting spacer sequence (SEQ ID NO: 11) for additional evaluation of cleavage and collateral activities of the Cas13f mutants. The RHO-targeting guide RNA consisting of 5′-DR sequence (SEQ ID NO: 2)—RHO-targeting spacer sequence (SEQ ID NO: 11)-DR sequence (SEQ ID NO: 2)-3′ is set forth in SEQ ID NO: 17.

### Transfection and Detection:

According to standard cell culture methods, HEK293T cells were grown in 24-well tissue culture plates to a suitable density before the cells were transfected with the two plasmids using a PEI transfection reagent. Transfected cells were cultured at 37° C. in an incubator under 5% CO2 for about 72 hours, before measuring EGFP and mCherry fluorescent signals in the cells with FACS. Low EGFP mean fluorescent intensity (MFI) indicated high cleavage activity as desired. High mCherry MI indicated low collateral cleavage activity as desired.

As a negative control (“NT”), an expression plasmid encoding F40S23 and a gRNA comprising a non-targeting spacer sequence (SEQ ID NO: 9) in place of the targeting spacer sequence was used with the reporter plasmid for transfection. Since the collateral cleavage is only trigged by on-target cleavage, theoretically neither collateral cleavage nor on-target cleavage should happen when a non-targeting spacer sequence is used. Therefore, all the MFI results (mean+SD) of the Cas13f mutants were normalized to the negative control.

As a positive control (“PT” or “F40S23”), an expression plasmid encoding F40S23 and a gRNA comprising a targeting spacer sequence was used with the reporter plasmid for transfection.

In addition, RT-qPCR was carried out for testing on an endogenous genome locus, SOD1, in Cos7 cells to investigate SOD1 mRNA knockdown indicative of cleavage activities of the Cas13f mutants. According to standard cell culture methods, Cos7 cells were grown in 6-well tissue culture plates to a suitable density before the cells were transfected with each of the expression plasmids encoding a Cas13f mutant and a SOD1-targeting guide RNA (SEQ ID NO: 18) using a PEI transfection reagent. After 72 hours, an amount of the top 30% mCherry-positive cells were sorted by flow sorting, total RNA was extracted from the positive cells, and SOD1 mRNA level was measured by RT-qPCR and normalized to a housekeeping gene, GAPDH.

The cleavage activity of each tested Cas13f mutant was inversely correlated to the EGFP MI. The lower the EGFP MI is, the higher the cleavage activity would be. Therefore, the cleavage activity of the Cas13f mutant relative to the reference F40S23 is shown as the reciprocal of EGFP MI of Cas13f mutant relative to EGFP MI of F40S23.

$$Cleavage activity relative to F 40 S23 = 1 / ( EGFP MFI of Cas 13 f mutant / EGFPMFI of F 40 S 23 ) ) .$$

The collateral activity of each tested Cas13f mutant was inversely correlated to the mCherry MI. The higher the mCherry MI is, the lower the collateral activity would be. Therefore, the collateral activity of the Cas13f mutant relative to the reference F40S23 is shown as the reciprocal of mCherry MI of Cas13f mutant relative to mCherry MEI of F40S23.

$$Collateral activity relative to F 40 S23 = 1 / ( mCherry MFI of Cas 13 f mutant / mCherry MFI of F 40 S 23 ) ) .$$

In addition, for the RT-qPCR test, the cleavage activity of each tested Cas13f mutant was inversely correlated to the SOD1 mRNA level. The lower the SOD1 mRNA level is, the higher the cleavage activity would be. Therefore, the cleavage activity of the Cas13f mutant relative to the reference F40S23 is shown as the reciprocal of SOD1 mRNA level of Cas13f mutant relative to SOD1 mRNA level of F40S23.

Cleavage activity relative to F40S23=1/(SOD1 mRNA level of Cas13f mutant/SOD1 mRNA level of F40S23)).

### Results

It was noted that the listed Cas13f mutants in Table 6 have not only higher cleavage activity but also lower collateral cleavage activity than F40S23.

TABLE 6
Averaged cleavage and collateral activities of
the listed Cas13f mutants, as presented by MFIs
with gRNA targeting ATXN2 RNA transcript (n = 3)

| | | Collateral | | Cleavage |
|---|---|---|---|---|
| | mCherry MFI | activity | EGFP MFI | activity |
| | relative | relative to | relative | relative to |
| Mutant | to NT | F40S23 | to NT | F40S23 |
| NT | 1.000 | — | 1.000 | — |
| F40S23 | 0.781 | 100.00% | 0.590 | 100.00% |
| F40S23 + D160A | 0.908 | 86.01% | 0.449 | 131.40% |
| F40S23 + T161A | 1.078 | 72.45% | 0.454 | 129.96% |
| F40S23 + L641A | 1.097 | 71.19% | 0.424 | 139.15% |
| F40S23 + T647A | 1.122 | 69.61% | 0.456 | 129.39% |
| F40S23 + P667A | 1.060 | 73.68% | 0.440 | 134.09% |
| F40S23 + D762A | 1.156 | 67.56% | 0.403 | 146.40% |
| F40S23 + A763V | 1.003 | 77.87% | 0.579 | 101.90% |

In addition, the RT-qPCR results in Table 7 below show the improved SOD1 mRNA knockdown efficiency of all the listed Cas13f mutants than F40S23.

TABLE 7
Averaged SOD1 mRNA level in Cos7 cells by RT-
qPCR for the listed Cas13f mutants (n = 3)

| | Averaged SOD1 | |
|---|---|---|
| | mRNA level | Cleavage activity |
| Mutant | relative to NT | relative to F40S23 |
| NT | 1.001 | — |
| F40S23 | 0.562 | 100.00% |
| F40S23 + D160A | 0.233 | 241.20% |
| F40S23 + T161A | 0.285 | 197.19% |
| F40S23 + L631A | 0.221 | 254.30% |
| F40S23 + L634A | 0.197 | 285.28% |
| F40S23 + H638A | 0.208 | 270.19% |
| F40S23 + L641A | 0.171 | 328.65% |
| F40S23 + D642A | 0.153 | 367.32% |
| F40S23 + T647A | 0.166 | 338.55% |
| F40S23 + P667A | 0.218 | 257.80% |
| F40S23 + V670A | 0.208 | 270.19% |
| F40S23 + D762A | 0.189 | 297.35% |

The above results show that the additional introduction of a single-point mutation listed in Table 5 into F40S23 enhanced the cleavage activity while maintaining or even lowering the collateral activity of F40S23.

Based on the above results and with the same experimental procedures, the single mutations were subsequently combined in pair for introduction into F40S23 for further evaluation of the cleavage and collateral activities of the resulting mutants, as shown in Table 8 below.

TABLE 8
Averaged cleavage and collateral activities of the listed Cas13f mutants
as presented by MFI with gRNA targeting RHO RNA transcript (n = 3)

| | | Collateral | | Cleavage |
|---|---|---|---|---|
| | mCherry MFI | activity | EGFP MFI | activity |
| | relative | relative to | relative | relative to |
| Mutant | to NT | F40S23 | to NT | F40S23 |
| NT | 1.000 | — | 1.000 | — |
| F40S23 | 0.869 | 100.00% | 0.665 | 100.00% |
| F40S23 + D160A | 0.892 | 97.42% | 0.578 | 115.05% |
| F40S23 + D642A | 1.084 | 80.17% | 0.497 | 133.80% |
| F40S23 + L631A | 0.921 | 94.35% | 0.533 | 124.77% |
| F40S23 + P667A | 0.964 | 90.15% | 0.528 | 125.95% |
| F40S23 + H638A | 0.913 | 95.18% | 0.540 | 123.15% |
| F40S23 + L634A | 0.956 | 90.90% | 0.620 | 107.26% |
| F40S23 + L641A | 1.058 | 82.14% | 0.636 | 104.56% |
| F40S23 + L631A&H638A | 0.978 | 88.85% | 0.640 | 103.91% |
| F40S23 + L631A&L641A | 1.055 | 82.37% | 0.840 | 79.17% |
| F40S23 + L631A&D642A | 0.966 | 89.96% | 0.655 | 101.53% |
| F40S23 + D160A&L631A | 0.968 | 89.77% | 0.469 | 141.79% |
| F40S23 + H638A&L641A | 0.909 | 95.60% | 0.700 | 95.00% |
| F40S23 + H638A&D642A | 0.921 | 94.35% | 0.464 | 143.32% |
| F40S23 + L641A&D642A | 0.995 | 87.34% | 0.551 | 120.69% |
| F40S23 + D160A&D642A | 1.113 | 78.08% | 0.430 | 154.65% |

The results in Table 8 shows that the listed Cas13f mutants have decreased collateral activity than F40S23, and the listed Cas13f mutants have at least 75% cleavage activity of F40S23 (e.g., F40S23+L631A&L641A), at least 95% cleavage activity of F40S23 (e.g., F40S23+H638A&L641A), or increased cleavage activity than F40S23.

Particularly, the mutant F40S23+D 160A&D642A (Cas13f-D160A+D642A+Y666A+Y677A, with its full length amino acid sequence set forth in SEQ ID NO: 4, designated as “hfCas13f”) achieved both the highest cleavage activity and the lowest collateral activity.

Additional evaluation was conducted to verify the cleavage and collateral activities of particular mutants in Tables 9-11.

TABLE 9
Averaged cleavage and collateral cleavage activities of Cas13f
mutants as presented by MFIs with gRNA targeting EGFP RNA
transcript (gRNA set forth in SEQ ID NO: 15) (n = 3)

| | | Collateral | | Cleavage |
|---|---|---|---|---|
| | mCherry MFI | activity | EGFP MFI | activity |
| | relative | relative to | relative | relative to |
| Mutant | to NT | F40S23 | to NT | F40S23 |
| NT | 1.000 | — | 1.000 | — |
| F40S23 | 1.024 | 100.00% | 0.374 | 100.00% |
| F40S23 + D160A&D642A | 0.982 | 104.28% | 0.283 | 132.16% |
| F40S23 + H638A&D642A | 0.957 | 107.00% | 0.284 | 131.69% |

The results in Table 9 show that for EGFP target, the mutants have significantly improved cleavage activity while maintaining the collateral activity, as compared to F40S23.

TABLE 10
Averaged cleavage and collateral cleavage activities of Cas13f
mutants as presented by MFIs with gRNA targeting ATXN2 RNA
transcript (gRNA set forth in SEQ ID NO: 16) (n = 3)

| | | Collateral | | Cleavage |
|---|---|---|---|---|
| | mCherry MFI | activity | EGFP MFI | activity |
| | relative | relative to | relative | relative to |
| Mutant | to NT | F40S23 | to NT | F40S23 |
| NT | 1.000 | — | 1.000 | — |
| F40S23 | 0.891 | 100.00% | 0.510 | 100.00% |
| F40S23 + D160A&D642A | 1.335 | 66.74% | 0.202 | 252.48% |
| F40S23 + H638A&D642A | 1.338 | 66.59% | 0.225 | 226.67% |

The results in Table 10 show that for ATXN2 target, the mutants have significantly improved cleavage activity and significantly reduced collateral activity, as compared to F40S23.

TABLE 11
Averaged SOD1 mRNA level in Cos7 cells by RT-qPCR for Cas13f
mutants (gRNA set forth in SEQ ID NO: 18) (n = 3)

| | Averaged | |
|---|---|---|
| | SOD1 mRNA | Cleavage |
| | level | activity |
| | relative | relative |
| Protein | to NT | to F40S23 |
| NT | 1.005 | — |
| F40S23 | 0.307 | 100.00% |
| F40S23 + D160A&D642A | 0.125 | 245.60% |
| F40S23 + H638A&D642A | 0.202 | 151.98% |

The results in Table 11 show that for SOD 1 target, the mutants have significantly improved cleavage activity as compared to mutant F40S23.

### Example 3 Engineering of hfCas13f for Increased Cleavage Activity

This Example demonstrates that by introducing a specific amino acid mutation into hfCas13f, the cleavage activity can be further substantially increased.

### Designs and Constructions:

To obtain a Cas13f mutant with increased cleavage activity, one of the non-basic amino acids of hfCas13f except those in the HEPN1 and HEPN2 domains was mutated to arginine (R, a common positively charged basic amino acid) to create Cas13f mutants (FIG. 5).

A two-plasmid mammalian fluorescence reporter system (FIG. 6) comprising a mutant encoding plasmid and a gRNA encoding plasmid was constructed for detection of the cleavage activities of the Cas13f mutants further engineered based on hfCas13f.

The mutant encoding plasmid comprised (1) a mCherry red fluorescent reporter gene (with its RNA transcript as an RNA target for cleavage activity of the Cas13f mutants) under the regulation of a SV40 promoter and a poly A sequence, (2) a Cas13f mutant coding sequence flanked by both 5′ and 3′ SV40 NLS (SEQ ID NO: 5) coding sequence under the regulation of a Cbh promoter and a poly A sequence, and (3) a BFP fluorescent reporter gene under the regulation of a CMV promoter and a poly A sequence. The blue fluorescence from BFP would indicate successful transfection and expression of the mutant encoding plasmid in host cells.

The gRNA encoding plasmid comprised a sequence encoding a mCherry-targeting gRNA (SEQ ID NO: 19) consisting of 5′-DR sequence (SEQ ID NO: 2)-mCherry-targeting spacer sequence (SEQ ID NO: 13)-DR sequence (SEQ ID NO: 2)-3′ under the regulation of a U6 promoter.

### Transfection and Detection:

HEK293T cells were cultured in 24-well tissue culture plates according to standard methods for 12 hours, before the two plasmids were co-transfected into the cells using standard polyethyleneimine (PEI) transfection. The transfected cells were then cultured at 37° C. under 5% CO₂ for about 48 hours. Then the BFP positive cultured cells were analyzed by flow cytometry.

As a negative control (“NT”), a mutant encoding plasmid encoding hfCas13f and a gRNA encoding plasmid encoding a non-targeting spacer sequence (SEQ ID NO: 14) in place of the mCherry-targeting spacer sequence (SEQ ID NO: 13) were used for transfection. All the mCherry (RFP) MFI results (mean±SD) of the Cas13f mutants were normalized to the negative control.

As a positive control (“PT” or “hfCas13f”), a mutant encoding plasmid encoding hfCas13f and a gRNA encoding plasmid encoding a mCherry targeting spacer sequence (SEQ ID NO: 13) were used for transfection.

The cleavage activity of each tested Cas13f mutant was inversely correlated to the mCherry MFI. The lower the mCherry MFI is, the higher the cleavage activity would be. Therefore, the cleavage activity of the Cas13f mutant relative to the reference hfCas13f is shown as the reciprocal of mCherry MFI of Cas13f mutant relative to mCherry MFI of hfCas13f.

Cleavage activity relative to hfCas13f=1/(mCherry MFI of Cas13f mutant/mCherry MFI of hfCas13f)).

### Results

The Cas13f mutants were tested in four batches with hfCas13f as a positive control, thereby excluding the effect of transfection efficiency on cleavage activity. The flow cytometry results show the mCherry MFI of each Cas13f mutant with a single amino acid substitution to R. Among others, the Cas13f mutants each with a single amino acid substitution to R at a position 183, 189, 200, 202, 204, 205, 213, 214, 222, 233, 239, 240, 241, 258, 259, 276, 282, 283, 298, 299, 300, 314, 320, 329, 338, 339, 345, 353, 361, 383, 410, 433, 451, 455, 497, 508, 509, 518, 520, 526, 574, 595, 598, 599, or 601 (as highlighted in grey) had weaker mCherry MFIs in one or more batches than that of hfCas13f, indicating increased cleavage activities (Table. 12-15).

TABLE 12
Averaged mCherrv MFI (n = 2) for Cas13f mutants

| | Averaged | Cleavage | | Averaged | Cleavage | | Averaged | Cleavage |
|---|---|---|---|---|---|---|---|---|
| | mCherry | activity | | mCherry | activity | | mCherry | activity |
| | MFI | relative | | MFI | relative | | MFI | relative |
| | relative | to | | relative | to | | relative | to |
| Mutant | to NT | hfCas13f | Mutant | to NT | hfCas13f | Mutant | to NT | hfCas13f |
| NT | 1.000 | — | I204R | 0.286 | 106.29% | G282R | 0.212 | 143.40% |
| hfCas13f | 0.304 | 100.00% | E205R | 0.273 | 111.36% | E283R | 0.276 | 110.14% |
| F169R | 1.079 | 28.17% | D212R | 0.376 | 80.85% | L289R | 0.994 | 30.58% |
| T170R | 0.490 | 62.04% | G214R | 0.257 | 118.29% | A292R | 0.346 | 87.86% |
| Y171R | 0.668 | 45.51% | G216R | 0.343 | 88.63% | L294R | 0.495 | 61.41% |
| E183R | 0.276 | 110.14% | A223R | 0.807 | 37.67% | I295R | 0.327 | 92.97% |
| F188R | 1.339 | 22.70% | L233R | 0.252 | 120.63% | Q298R | 0.298 | 102.01% |
| L189R | 0.231 | 131.60% | N269R | 0.511 | 59.49% | D299R | 0.263 | 115.59% |
| Q200R | 0.246 | 123.58% | F272R | 1.110 | 27.39% | A300R | 0.295 | 103.05% |
| D201R | 0.346 | 87.86% | E273R | 0.330 | 92.12% | N301R | 0.311 | 97.75% |
| D202R | 0.286 | 106.29% | G276R | 0.289 | 105.19% | | | |

TABLE 13
Averaged mCherry MFI (n = 2 or 1) for Cas13f mutants

| | | Cleavage | | | Cleavage | | | Cleavage |
|---|---|---|---|---|---|---|---|---|
| | | activity | | | activity | | | activity |
| | Averaged | relative | | Averaged | relative | | Averaged | relative |
| | mCherry | to | | mCherry | to | | mCherry | to |
| Mutant | MFI | hfCas13f | Mutant | MFI | hfCas13f | Mutant | MFI | hfCas13f |
| NT | 1.000 | | Y372R | 0.534 | 80.15% | N552R | 0.486 | 88.07% |
| hfCas13f | 0.428 | 100.00% | Y400R | 0.515 | 83.11% | P557R | 0.469 | 91.26% |
| T240R | 0.374 | 114.44% | N474R | 0.460 | 93.04% | F560R | 0.667 | 64.17% |
| Y241R | 0.395 | 108.35% | D475R | 0.544 | 78.68% | I565R | 0.659 | 64.95% |
| V303R | 0.534 | 80.15% | N481R | 0.544 | 78.68% | L566R | 0.504 | 84.92% |
| G305R | 0.549 | 77.96% | E494R | 0.499 | 85.77% | S571R | 0.699 | 61.23% |
| T308R | 0.457 | 93.65% | E495R | 0.477 | 89.73% | V574R | 0.379 | 112.93% |
| Q309R | 0.472 | 90.68% | T510R | 0.447 | 95.75% | E584R | 0.774 | 55.30% |
| F310R | 0.504 | 84.92% | L515R | 0.491 | 87.17% | E590R | 0.453 | 94.48% |
| F314R | 0.336 | 127.38% | Q520R | 0.330 | 129.70% | D603R | 0.677 | 63.22% |
| N316R | 0.504 | 84.92% | I522R | 0.527 | 81.21% | D605R | 0.594 | 72.05% |
| A317R | 0.489 | 87.53% | G531R | 0.531 | 80.60% | M607R | 0.501 | 85.43% |
| Q321R | 0.489 | 87.53% | I532R | 0.648 | 66.05% | L613R | 0.459 | 93.25% |
| Q322R | 0.509 | 84.09% | N536R | 0.590 | 72.54% | Y614R | 0.487 | 87.89% |
| E327R | 0.525 | 81.52% | L537R | 1.127 | 37.98% | E615R | 0.707 | 60.54% |
| L329R | 0.364 | 117.58% | T540R | 0.586 | 73.04% | N617R | 0.506 | 84.58% |
| E332R | 0.565 | 75.75% | S546R | 0.647 | 66.15% | | | |

TABLE 14
Averaged mCherry MFI (n = 2) for Cas13f mutants

| | | Cleavage | | | Cleavage | | | Cleavage |
|---|---|---|---|---|---|---|---|---|
| | | activity | | | activity | | | activity |
| | Averaged | relative | | Averaged | relative | | Averaged | relative |
| | mCherry | to | | mCherry | to | | mCherry | to |
| Mutant | MFI | hfCas13f | Mutant | MFI | hfCas13f | Mutant | MFI | hfCas13f |
| NT | 1.000 | | A340R | 0.316 | 75.32% | T433R | 0.212 | 112.26% |
| hfCas13f | 0.238 | 100.00% | G345R | 0.225 | 105.78% | V440R | 0.260 | 91.54% |
| M236R | 0.731 | 32.56% | I347R | 0.253 | 94.07% | L451R | 0.221 | 107.69% |
| F238R | 0.915 | 26.01% | D349R | 0.246 | 96.75% | F452R | 0.312 | 76.28% |
| Y239R | 0.237 | 100.42% | L352R | 0.260 | 91.54% | L455R | 0.206 | 115.53% |
| Q242R | 0.253 | 94.07% | N353R | 0.230 | 103.48% | E580R | 0.575 | 41.39% |
| Q249R | 0.282 | 84.40% | G367R | 0.257 | 92.61% | A581R | 0.479 | 49.69% |
| E252R | 0.289 | 82.35% | I370R | 0.294 | 80.95% | S595R | 0.198 | 120.20% |
| D258R | 0.199 | 119.60% | E410R | 0.163 | 146.01% | F598R | 0.168 | 141.67% |
| I259R | 0.182 | 130.77% | F418R | 0.252 | 94.44% | F599R | 0.202 | 117.82% |
| W262R | 0.479 | 49.69% | T420R | 0.693 | 34.34% | Q600R | 0.241 | 98.76% |
| Q267R | 0.458 | 51.97% | Y426R | 0.471 | 50.53% | S601R | 0.201 | 118.41% |
| F339R | 0.195 | 122.05% | P428R | 0.374 | 63.64% | G612R | 0.301 | 79.07% |

TABLE 15
Averaged mCherry MFI (n = 2 or 1) for Cas13f mutants

| | | Cleavage | | | Cleavage | | | Cleavage |
|---|---|---|---|---|---|---|---|---|
| | | activity | | | activity | | | activity |
| | Averaged | relative | | Averaged | relative | | Averaged | relative |
| | mCherry | to | | mCherry | to | | mCherry | to |
| Mutant | MFI | hfCas13f | Mutant | MFI | hfCas13f | Mutant | MFI | hfCas13f |
| NT | 1.000 | | E341R | 0.325 | 98.46% | Q508R | 0.263 | 121.67% |
| hfCas13f | 0.320 | 100.00% | N356R | 0.422 | 75.83% | I509R | 0.313 | 102.24% |
| S192R | 0.406 | 78.82% | S361R | 0.316 | 101.27% | M518R | 0.245 | 130.61% |
| Y203R | 0.369 | 86.72% | M379R | 0.782 | 40.92% | T523R | 0.381 | 83.99% |
| I213R | 0.253 | 126.48% | N383R | 0.316 | 101.27% | L526R | 0.226 | 141.59% |
| I222R | 0.303 | 105.61% | L386R | 0.703 | 45.52% | E533R | 0.776 | 41.24% |
| P265R | 0.617 | 51.86% | Y397R | 0.596 | 53.69% | L535R | 0.421 | 76.01% |
| C290R | 0.635 | 50.39% | N436R | 0.324 | 98.77% | D542R | 0.363 | 88.15% |
| V320R | 0.294 | 108.84% | A444R | 0.389 | 82.26% | A549R | 0.399 | 80.20% |
| N337R | 0.945 | 33.86% | D478R | 0.416 | 76.92% | | | |
| Y338R | 0.227 | 140.97% | D497R | 0.317 | 100.95% | | | |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. An engineered Cas 13 f polypeptide, wherein the engineered Cas 13 f polypeptide:
(1) has a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.6%, 99.7%, or 99.8%) and less than 100% to the amino acid sequence of SEQ ID NO: 3;
(2) comprises a double mutation corresponding to the double mutation Y666A and Y677A of the amino acid sequence of SEQ ID NO: 3; and
(3) has an increased spacer sequence-specific cleavage activity compared to that of the amino acid sequence of SEQ ID NO: 3 and/or a decreased spacer sequence-independent collateral cleavage activity compared to that of the amino acid sequence of SEQ ID NO: 3.

2. The engineered Cas13f polypeptide of claim 1, having at least about 70% (e.g., at least about 70%, 75%, 80%, 85%, 90%, 95%, 100%, 105%, 110%, 115%, 120%, 125%, 130%, 135%, 140%, 145%, or 150%) of the spacer sequence-specific cleavage activity compared to that of the amino acid sequence of SEQ ID NO: 3.

3. The engineered Cas13f polypeptide of claim 1 or 2, having at most about 120% (e.g., at most about 120%, 115%, 110%, 105%, 100%, 95%, 90%, 85%, 80%, 75%, or 70%) spacer sequence-independent collateral cleavage activity compared to that of the amino acid sequence of SEQ ID NO: 3.

4. The engineered Cas13f polypeptide of any of claims 1-3, having (1) at least about 75% of the spacer sequence-specific cleavage activity compared to that of the amino acid sequence of SEQ ID NO: 3, and (2) at most about 90% spacer sequence-independent collateral cleavage activity compared to that of the amino acid sequence of SEQ ID NO: 3.

5. The engineered Cas13f polypeptide of any of claims 1-3, having (1) at least about 100% of the spacer sequence-specific cleavage activity compared to that of the amino acid sequence of SEQ ID NO: 3, and (2) at most about 90% spacer sequence-independent collateral cleavage activity compared to that of the amino acid sequence of SEQ ID NO: 3.

6. The engineered Cas13f polypeptide of any of claims 1-3, having (1) at least about 130% of the spacer sequence-specific cleavage activity compared to that of the amino acid sequence of SEQ ID NO: 3, and (2) at most about 110% spacer sequence-independent collateral cleavage activity compared to that of the amino acid sequence of SEQ ID NO: 3.

7. The engineered Cas13f polypeptide of any of claims 1-3, having (1) at least about 130% of the spacer sequence-specific cleavage activity compared to that of the amino acid sequence of SEQ ID NO: 3, and (2) at most about 100% spacer sequence-independent collateral cleavage activity compared to that of the amino acid sequence of SEQ ID NO: 3.

8. The engineered Cas13f polypeptide of any of claims 1-3, having (1) at least about 130% of the spacer sequence-specific cleavage activity compared to that of the amino acid sequence of SEQ ID NO: 3, and (2) at most about 90% spacer sequence-independent collateral cleavage activity compared to that of the amino acid sequence of SEQ ID NO: 3.

9. The engineered Cas13f polypeptide of any of claims 1-8, comprising an amino acid substitution at one or more positions corresponding to positions selected from the group consisting of positions 160, 161, 183, 189, 200, 202, 204, 205, 213, 214, 222, 233, 239, 240, 241, 258, 259, 276, 282, 283, 298, 299, 300, 314, 320, 329, 338, 339, 345, 353, 361, 383, 410, 433, 451, 455, 497, 508, 509, 518, 520, 526, 574, 595, 598, 599, 601, 631, 634, 638, 641, 642, 647, 667, 670, 762, 763, and combinations thereof of the amino acid sequence of SEQ ID NO: 3.

10. The engineered Cas 13f polypeptide of claim 9, wherein the amino acid substitution is a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F), or a positively charged amino acid residue (such as, Lysine (Lys/K), Arginine (Arg/R), Histidine (His/H)).

11. The engineered Cas 13f polypeptide of claim 10, wherein the amino acid substitution is a substitution of a non-Arginine (Arg/R) residue with an Arginine (Arg/R) residue.

12. The engineered Cas13f polypeptide of claim 10, wherein the amino acid substitution is a substitution of a non-Alanine (Ala/A) residue with an Alanine (Ala/A) residue.

13. The engineered Cas 13f polypeptide of claim 10, wherein the amino acid substitution is a substitution of an Alanine (Ala/A) residue with a Valine (Val/V) residue.

14. The engineered Cas13f polypeptide of any of claims 1-13, comprising an amino acid substitution at one or more positions corresponding to positions selected from the group consisting of positions 160, 161, 631, 634, 638, 641, 642, 647, 667, 670, 762, 763, and combinations thereof of the amino acid sequence of SEQ ID NO: 3.

15. The engineered Cas 13f polypeptide of claim 14, wherein the amino acid substitution is a substitution of a non-Alanine (Ala/A) residue with an Alanine (Ala/A) residue or an Alanine (Ala/A) residue with a Valine (Val/V) residue.

16. The engineered Cas13f polypeptide of claim 14 or 15, comprising an amino acid substitution with an Alanine (Ala/A) residue at one or more positions corresponding to positions selected from the group consisting of positions D160, H638, D642, and combinations thereof of the amino acid sequence of SEQ ID NO: 3.

17. The engineered Cas 13f polypeptide of claim 16, comprising an amino acid substitution with an Alanine (Ala/A) residue at one or more positions corresponding to:
(a) position D 160,
(b) position H638,
(c) position D642,
(d) positions D160 and H638,
(e) positions D160 &D642,
(f) positions H638 &D642, or
(g) positions D160 & L631,
of the amino acid sequence of SEQ ID NO: 3.

18. The engineered Cas13f polypeptide of claim 17 comprising a quadruple amino acid substitution with Alanine (Ala/A) residues at positions corresponding to positions D160, D642, Y666, and Y677 of the amino acid sequence of SEQ ID NO: 1.

19. The engineered Cas13f polypeptide of claim 18, comprising, consisting essentially of, or consisting of the amino acid sequence of SEQ ID NO: 4.

20. The engineered Cas 13f polypeptide of any of claims 1-19, comprising an amino acid substitution at one or more positions corresponding to positions selected from the group consisting of positions 183, 189, 200, 202, 204, 205, 213, 214, 222, 233, 239, 240, 241, 258, 259, 276, 282, 283, 298, 299, 300, 314, 320, 329, 338, 339, 345, 353, 361, 383, 410, 433, 451, 455, 497, 508, 509, 518, 520, 526, 574, 595, 598, 599, 601, and combinations thereof of the amino acid sequence of SEQ ID NO: 3.

21. The engineered Cas 13f polypeptide of claim 20, wherein the amino acid substitution is a substitution of a non-Arginine (Arg/R) residue with an Arginine (Arg/R) residue.

22. The engineered Cas13f polypeptide of claim 21, having an increased spacer sequence-specific cleavage activity compared to that of the amino acid sequence of SEQ ID NO: 4.

23. The engineered Cas13f polypeptide of claim 22, having at least about 105%, 110%, 115%, 120%, 125%, 130%, 135%, 140%, 145%, or 150% of the spacer sequence-specific cleavage activity compared to that of the amino acid sequence of SEQ ID NO: 4.

24. The engineered Cas 13f polypeptide of any of claims 20-23, comprising an amino acid substitution with an Arginine (Arg/R) residue at one or more positions corresponding to positions selected from the group consisting of positions G282, F314, Y338, E410, Q520, L526, F598, and combinations thereof of the amino acid sequence of SEQ ID NO: 3.

25. A polynucleotide encoding the engineered Cas13f polypeptide of any of claims 1-24; optionally the polynucleotide is codon optimized for expression in a eukaryote, a mammal, such as, a non-human mammal, a non-human primate, a human, a plant, an insect, a bird, a reptile, a rodent (e.g., mouse, rat), a fish, a nematode, or a yeast.

26. A CRISPR-Cas13f system comprising:
a) the engineered Cas13f polypeptide of any of claims 1-24 or a polynucleotide (e.g., a DNA, an RNA) encoding the engineered Cas 13 f polypeptide; and
b) a guide nucleic acid or a polynucleotide (e.g., a DNA or an RNA) encoding the guide nucleic acid, the guide nucleic acid comprising:
i. a direct repeat (DR) sequence capable of forming a complex with the engineered Cas13f polypeptide; and,
ii. a spacer sequence capable of hybridizing to a target RNA, thereby guiding the complex to the target RNA;
optionally wherein the DR sequence has substantially the same secondary structure as the secondary structure of the DR sequence of SEQ ID NO: 2; and
optionally wherein the spacer sequence is in a length of at least about 15 nucleotides, optionally about 30 nucleotides.

27. A vector comprising the polynucleotide of claim 25;
optionally wherein the polynucleotide is operably linked to a promoter and optionally an enhancer;
optionally wherein the promoter is a constitutive promoter, an inducible promoter, a ubiquitous promoter, or a cell, tissue, or organ specific promoter;
optionally wherein the vector is a plasmid;
optionally wherein the vector is a retroviral vector, a phage vector, an adenoviral vector, a herpes simplex viral (HSV) vector, an AAV vector, or a lentiviral vector;
optionally wherein the AAV vector is a recombinant AAV particle comprising a capsid with a serotype of AAV1, AAV2, AAV3A, AAV3B, AAV4, AAV5, AAV6, AAV7, AAVrh74, AAV8, AAV9, AAV10, AAV11, AAV12, AAV13, AAV-DJ, AAV.PHP. eB, a member of the Clade to which any of the AAV1-AAV13 belong, or a functional variant (e.g., a functional truncation) thereof; and/or
optionally wherein the AAV vector is an RNA-encapsulated AAV particle.

28. A delivery system comprising (1) a delivery vehicle, and (2) the engineered Cas13f polypeptide of any of claims 1-24, the polynucleotide of claim 25, the CRISPR-Cas13f system of claim 26, or the vector of claim 27;
optionally wherein the delivery vehicle is a nanoparticle (e.g., LNP), a liposome, an exosome, a microvesicle, or a gene-gun.

29. A method of modifying a target RNA, comprising contacting the target RNA with the CRISPR-Cas13f system of claim 26, the vector of claim 27, or the delivery system of claim 28, thereby modifying the target RNA.

30. A method of treating a disease in a subject in need thereof, comprising administering to the subject the CRISPR-Cas13f system of claim 26, wherein the disease is associated with a target RNA, wherein the CRISPR-Cas 13 f system modifies the target RNA, and wherein the modification of the target RNA treats the disease.

31. The method of claim 29 or 30, wherein the target RNA is mRNA, a tRNA, a ribosomal RNA (rRNA), a microRNA (miRNA), a non-coding RNA, a long non-coding (lnc) RNA, a nuclear RNA, an interfering RNA (iRNA), a small interfering RNA (siRNA), a ribozyme, a riboswitch, a satellite RNA, a microswitch, a microzyme, or a viral RNA;
optionally wherein the target RNA is encoded by a eukaryotic DNA; and/or
optionally wherein the eukaryotic DNA is a mammal DNA, such as a non-human mammalian DNA, a non-human primate DNA, a human DNA, a plant DNA, an insect DNA, a bird DNA, a reptile DNA, a rodent (e.g., mouse, rat) DNA, a fish DNA, a nematode DNA, or a yeast DNA.

32. The method of claim 30 or 31, wherein the disease is selected from the group consisting of glaucoma, age-related RGC loss, optic nerve injury, retinal ischemia, Leber's hereditary optic neuropathy, a neurological condition associated with degeneration of RGC neurons, a neurological condition associated with degeneration of functional neurons in the striatum of a subject in need thereof, Parkinson's disease, Alzheimer's disease, Huntington's disease, Schizophrenia, depression, drug addiction, movement disorder such as chorea, choreoathetosis, and dyskinesias, bipolar disorder, Autism spectrum disorder (ASD), dysfunction, MECP2 duplication syndrome (MDS), Angelman syndrome, age-related macular degeneration (AMD), and Amyotrophic Lateral Sclerosis (ALS).
